(19) United States
(12) Patent Application Publication
(10) Pub. No.: US 2011/0086042 A1
(43) Pub. Date: Apr. 14, 2011

(54) Centrosomal Proteins, Nucleic Acids and Method of Use Thereof

(76) Inventor: Qingshen Gao, Lake Forest, IL (US)

(21) Appl. No.: 11/992,813

(22) PCT Filed: Oct. 2, 2006
(86) PCT No.: PCT/US2006/038325

§ 371 Date: Nov. 24, 2010

Related U.S. Application Data
(60) Provisional application No. 60/722,752, filed on Sep. 30, 2005.

(51) Int. Cl. A61K 39/395; C07K 14/00; C12N 15/00; C12N 15/63; C12N 5/00; C07K 16/00; A61K 31/713; C12Q 1/02; C12Q 1/68; A61P 35/00; A61P 21/00

(52) U.S. Cl. 424/172.1; 530/350; 536/23.1; 435/320.1; 435/325; 530/389.1; 530/387.9; 514/44 A; 435/29; 435/6

(57) ABSTRACT

The present invention provides novel isolated centrobin polynucleotides and polypeptides encoded by the centrobin polynucleotides. The invention additionally provides methods of inhibiting cell growth by contacting a cell with a centrobin inhibitor.

### FIELD OF THE INVENTION

The invention relates to polynucleotides and polypeptides encoded by such polynucleotides, as well as vectors, host cells, antibodies and recombinant methods for producing the polypeptides and polynucleotides. The nucleic acids and proteins of the present invention useful, for example, as target molecules for developing drugs against cancer.

### BACKGROUND OF THE INVENTION

Breast cancer is by far the most frequently diagnosed cancer in women. Each year over 186,000 new cases and 46,000 deaths are reported in United State alone. Germ-line mutations in the breast cancer susceptibility genes such as BRCA2 confer susceptibility to familial early-onset breast and ovarian cancers.

Studies indicated that BRCA2 plays an important role in DNA repair and the regulation of centrosome duplication. Centrosome defects are a common feature of malignant tumors. Uncoupling of centrosome duplication cycle and cell division cycle leads to an abnormal number of centrosomes per cell, i.e. centrosome amplification, which was frequently observed both in in vitro cultured tumor cells and a large variety of tumor tissues of different stages. Many tumor suppressors have been found to regulate centrosome duplication. However, the components and process of regulating centrosomal duplication remains largely unknown.

### SUMMARY OF THE INVENTION

The invention is based, in part, upon the discovery of polynucleotide sequences encoding a novel centrosomal protein, which interacts with BRCA-2. The polypeptides and nucleic acids are referred to herein as centrobin (Centrosomal BRCA2 interacting protein).

Accordingly, in one aspect the invention provides an isolated polypeptide that includes the amino acid sequence of SEQ ID NO: 2 or SEQ ID NO: 4 or fragment thereof. The polypeptide is 80%, 85%, 90%, 95%, 97%, 98% or 99% or more identical to the amino acid sequence of SEQ ID NO:2 or 4. Identity is measured by Clustal W. Alternatively identity is measured by methods known in the art such as FASTA or BLAST analysis.

A centrobin polypeptide fragment is less than 800, 700, 600, 550, 500, 450, 400, 300, or 200 amino acids in length. The polypeptide fragment has a centrobin activity. A centrobin activity is exemplified by binding to BRCA-2 or localizing to the centrosome. For example, a centrobin polypeptide fragment includes amino acids 371-903 of SEQ ID NO:2.

The invention further provides an isolated nucleic acid including the nucleotide sequence of SEQ ID NO 1 or 3 or a nucleic acid molecule that is complementary to the nucleic acid sequence of SEQ ID NO: 1 or 3. As used herein, the term “complementary” refers to Watson-Crick or Hoogsteen base pairing between nucleotide units of a nucleic acid molecule, and the term “binding” means the physical or chemical interaction between two polypeptides or compounds or associated polypeptides or compounds or combinations thereof. The nucleic acid encodes a polypeptide that is 80%, 85%, 90%, 95%, 97%, 98%, 99% or more identical to the amino acid sequence of SEQ ID NO:2 or 4. The nucleic acid encodes a polypeptide of SEQ ID NO:2 or 4. The nucleic acid is, e.g., a genomic DNA fragment, or a cDNA molecule.

Also included in the invention is a vector containing one or more of the nucleic acid molecules described herein, and a cell containing the vectors or nucleic acids described herein.

In a further aspect, the invention provides an antibody that binds specifically to a centrobin polypeptide or nucleic acid. The antibody is a, e.g., a monoclonal or polyclonal antibody, or fragments, homologs, analogs, and derivatives thereof. The invention also includes a composition including a centrobin polypeptide, nucleic acid or antibody. Optionally the composition further contains a carrier of diluent. The present invention is further directed to kits in one or more containers containing the composition described herein.

In another aspect, the invention provides methods of inhibiting cytokinesis cell growth by contacting a cell with a composition containing a centrobin inhibitor. By inhibiting cell growth is meant that the treated cell proliferates at a lower rate or has decreased viability than an untreated cell. By cytokinesis is meant division of the cytoplasm of a cell following division of the nucleus. Cell growth is determined by measuring DNA synthesis or by other by proliferation assays known in the art. For example, cell proliferation is measured by BrdU incorporation. A decrease in BrdU incorporation in the presence of the composition compared to the absence of the composition indicates that cell proliferation is inhibited. The cell is contacted with the composition in amount sufficient to induce apoptotic cell death. The cell is a cancer cell such as a breast cancer cell, a pancreatic cancer cell, a stomach cancer cell or a skin cancer cell. The cell is contacted in vivo, in vitro or ex vivo. The subject to be treated has been diagnosed as suffering from or predisposed to developing a cancer. For example, the patient is identified as having a tumor, a tissue/cell that is characterized as having an altered (e.g. abnormal) centrosome number compared to a normal, non-cancerous tissue/cell, or has a genetic predisposition to developing a tumor.

A symptom of cancer is also alleviated by administering to a subject a composition comprising a centrobin inhibitor. Alleviating a symptom of cancer leads to clinical benefit such as a reduction in expression of centrobin, or a decrease in size, prevalence, or metastatic potential of the tumor in the subject. Clinical benefit is determined in association with any known method for diagnosing or treating the particular tumor type. The methods of the invention also confer clinical benefits to genetic disorders, e.g., muscular dystrophy or a laminopathy (such as Ermery-Dreifuss muscular dystrophy type 2, familial partial lipodystrophy, limb girdle muscular dystrophy type 1B, dilated cardimyopathy, Charcot-Marie-Tooth disorder type 2B1, mandibuloacral dysplasia, childhood progeria syndrome (Hutchinson-Gilford syndrome) and a subset of Werner syndrome), which are characterized by aberrant expression or activity of centrobin.

The subject is a mammal such as human, a primate, mouse, rat, dog, cat, cow, horse, or pig. The subject is suffering from or at risk of developing cancer. The cancer is a breast cancer, pancreatic cancer, a stomach cancer or a skin cancer. A subject suffering from or at risk of developing cancer is identified by methods known in the art. For example, by the presence of a cancer marker, altered centrosome number, or by a biopsy.

A centrobin inhibitor is a compound which decreases the expression or activity of centrobin. For example, the compound is an antisense centrobin nucleic acid, a centrobin-specific short-interfering RNA, a centrobin antibody or a centrobin specific ribozyme. An exemplary centrobin siRNA includes SEQ ID NO: 5. The inhibitors, proteins, and nucleic acids derived herein are formulated as pharmaceutical compositions suitable for administration to a human. For example, the compositions are in the form of an intravenous fluid, an injectable compositions, a solid or semi-solid implantable composition, or a dermal patch.

Centrobin inhibitors preferentially induce apoptotic cell death of tumor cells compared to normal, non-cancerous cells. For example, a centrobin inhibitor leads to at least 10%, 20%, 50%, 2-fold, 5-fold, 10-fold or more death of tumor cells compared to normal cells. The effect of centrobin inhibitors on normal cells is arrest in cell cycle phase G1; normal cells then resume normal cell duplication processes in the absence of the inhibitor (e.g. after the inhibitor is removed or cleared by the body).

Optionally, the cell or subject is further contacted with an anti-proliferative agents such as a chemotherapeutic compound, prior to, following or concomitant with the centrobin inhibitor.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 demonstrates centrobin mRNA expression, amino acid sequence and structure. FIG. 1A is a photograph of a tissue Northern blot with 2 μg of polyA mRNA per lane (Clontech) using a ³²P-labeled centrobin probe followed by autoradiography. Hybridization with the 36B4 probe was used as a loading control. FIG. 1B is a photograph of a tissue Northern blot with 20 μg of total mRNA (Clontech) from the indicated breast cell lines using a ³²P-labeled centrobin probe followed by autoradiography. Hybridization with the 36B4 probe was used as a loading control. FIG. 1C indicates the predicted amino acid sequence of centrobin (SEQ ID NO: 2). FIG. 1D indicates 22 residues present in centrobin-β. (SEQ ID NO: 6). FIG. 1E is a schematic illustration of the coiled-coil regions of centrobin, ninein, and pericentrin as predicted by the DNASTAR program. Boxes indicate the coiled-coil region; lines indicate noncoiled regions. Centrobin-C designates the C-terminal fragment of centrobin that was isolated from a yeast two hybrid screen. FIG. 1F is a photograph of a Western blot demonstrating centrobin protein expression. Cell lysate from the indicated cell lines or 293T cells transfected with pCR3.1 vector, Myc-centrobin or GFP-centrobin constructs were fractionated by a 6% SDS-PAGE and blotted with affinity-purified anti-centrobin or anti-Myc antibodies.

FIG. 2 is a series of photographs demonstrating the localization of centrobin to the centrosomes. FIG. 2A shows that endogenous centrobin localized to the centrosomes. T47D, MCF-7, 76N, and Capan-1 cells were grown on coverslips, fixed with cold methanol, stained with affinity-purified anti-centrobin (1 μg/ml) and anti-α-tubulin (Sigma, 1:500) or anti-γ-tubulin (Sigma, 1:400), then stained with rhodamine-labeled goat anti-rabbit IgG and FITC-labeled goat anti-mouse IgG. DNA was stained with DAPI. Scale bar: 5 μm. FIG. 2B shows the localization of GFP-tagged centrobin to the centrosomes. 76NTert cells were grown on coverslips, transfected with pEGFP-Myc-centrobin, fixed with cold methanol, and stained with anti-γ-tubulin (Sigma, 1:400), then with rhodamine-labeled goat anti-mouse IgG. DNA was stained with DAPI. Scale bar: 5 μm. FIG. 2C shows the localization of Myc-tagged centrobin to the centrosomes. 76NTert cells were transfected with pCR3.1-Myc-centrobin, fixed with cold methanol, and stained with anti-Myc antibody 9E10 (1 μg/ml) and anti-γ-tubulin (Sigma, 1:400), then with rhodamine-labeled goat anti-mouse IgG and FITC-labeled goat anti-rabbit IgG. DNA was stained with DAPI. Scale bar: 5 μm. FIG. 2D shows the localization of Myc-tagged centrobin-C to the centrosomes. 76NTert cells were grown on coverslips, transfected with pSG5-Myc-centrobin-C, and fixed with cold methanol, then stained with anti-Myc and anti-γ-tubulin antibodies (Sigma, 1:400). Scale bar: 5 μm.

FIG. 3 is a series of photographs demonstrating the co-purification of centrobin with the centrosomes through sucrose gradients as shown by Western blotting. Centrosomes from 293T were purified through a 40%-70% sucrose gradient as described (Blomberg-Wirschell and Doxsey, 1998). The resulting fractions were separated on SDS-PAGE gels and blotted with anti-centrobin, anti-γ-tubulin, anti-Lamin B1, and anti-Cb1 antibodies.

FIG. 4 is a series of photographs showing that centrobin localized to the daughter centriole. FIG. 4A demonstrates the localization of centrobin during different phases of cell cycle in 76NTert cells. 76NTert cells were synchronized by mitotic shake-off and replating. Cells were harvested and briefly extracted, fixed with cold methanol, and stained with anti-centrobin (red) and anti-centrin-2 (green) antibodies. FIG. 4B shows centrobin localization in interphase NIH3T3 cells. The cells were fixed with cold methanol, then stained with anti-acetylated-α-tubulin (green) and anti-centrobin (red) antibodies. FIG. 4C shows centrobin localization in U2OS cells treated with HU. U2OS cells were treated with 16 mM HU for 72 h, fixed with cold methanol, and stained with anti-centrobin (red) and anti-centrin-2 (green) antibodies. FIG. 4D shows immunogold electron microscopic localization of centrobin on the daughter centrioles. 10-nm gold-conjugated anti-centrobin antibodies were detected on daughter centrioles but not mother centrioles. (M, mother centrioles, D, daughter centrioles)

FIG. 5 is a series of photographs demonstrating that centrobin depletion did not affect the localization of γ-tubulin or microtubule organization and nucleation. FIG. 5A is a set of photographs of a Western blot analysis of HeLa cells transfected with centrobin siRNA or control siRNA. HeLa cells were transfected with control (scrambled or FITC-GFP siRNA) or two-centrobin siRNA. After 72 h, cells were harvested and separated through a 6% SDS-PAGE, then blotted with anti-centrobin and anti-α-tubulin antibodies. FIG. 5B is a series of photographs of immunostaining of centrobin. HeLa cells transfected with scrambled or centrobin siRNA #1 were stained with anti-centrobin and then with rhodamine-labeled goat anti-rabbit IgG. DNA was stained with DAPI. FIG. 5C is a series of photographs of cells depleted of centroglobin using siRNA. Centrobin depletion did not affect the localization of γ-tubulin. HeLa cells transfected with scrambled or centrobin siRNA #1 were stained with anti-centrobin and anti-γ-tubulin, then with rhodamine-labeled goat anti-rabbit IgG and FITC-labeled goat anti-mouse IgG. FIG. 5D is a series of photographs showing that centrobin depletion did not affect microtubule organization and nucleation. HeLa cells were transfected with scrambled siRNA or centrobin siRNA; after 72 h, the cells were treated with 1 uM Nocodazole for 1 h, then washed three times with PBS to remove Nocodazole. The cells were harvested for fixation using cold methanol and stained for α-tubulin at 0, 2, 5, 10, and 15 minutes after the removal of Nocodazole.

FIG. 6 is a series of graphs and photographs showing that centrobin depletion inhibited centrosome duplication. FIG. 6A is a bar graph demonstrating that centrobin depletion inhibited centrosome duplication in interphase HeLa cells. HeLa cells were transfected with scrambled siRNA or centrobin siRNA and 72 h later, fixed with cold methanol, then stained with anti-centrobin and anti-centrin-2 antibodies. The number of centrioles was counted according to the centrin-2 staining in interphase cells with an undetectable level of centrobin. Data presented are the percentages of cells with >4, 4, 2, 1, or 0 centrioles (average from three independent experiments). For interphase cells, 300 cells were counted in every experiment. FIG. 6B is a bar graph demonstrating that centrobin depletion inhibited centrosome duplication in mitotic HeLa cells. HeLa cells were transfected with scrambled siRNA or centrobin siRNA and 72 h later, fixed with cold methanol, then stained with anti-centrobin and anti-centrin-2 antibodies. The number of centrioles was counted according to the centrin-2 staining in mitotic cells with an undetectable level of centrobin. Data presented are the percentages of cells with >4, 4, 2, 1, or 0 centrioles (average from three independent experiments). For mitotic cells, 100 cells were counted in every experiment. FIG. 6C is a series of photographs showing electron microscopic examination of centrosomes from control or centrobin depleted-HeLa cells over consecutive thick sections spanning the entire nuclear/centrosome complexes. A representative control cell with two centrioles and a centrobin depleted cell with one centriole were shown here. FIG. 6D is a bar graph showing that centrobin depletion inhibited centriole duplication in HeLa cells arrested by HU. HeLa cells were transfected with scrambled siRNA or centrobin siRNA. Forty-eight h later the cells were treated with 16 mM HU for another 24 h, pulse-labeled with BrdU for 30 min, then fixed and stained with anti-BrdU and anti-centrin. Data presented in FIG. 6D are the percentage of cells with 4, 2, 1, or 0 centrioles (average from three independent experiments with 300 cells counted in every experiment). FIG. 6E is a bar graphs showing an increase in BrdU negative cells in cultures treated with centrobin siRNA. Data presented in FIG. 6E are the percentages of cells with positive or negative BrdU staining (average from three independent experiments with 300 cells were counted in every experiment). FIG. 6F as a set of bar graphs showing that centrobin depletion inhibited centrosome overamplification in U2OS cells treated with HU. U2OS cells were either first treated with HU for 16 hours, then the cells were transfected with scrambled siRNA or centrobin siRNA, and incubated for additional 48 h in the presence of 16 mM HU(HU→siRNA); or first transfected with scrambled siRNA or centrobin siRNA, then 8 h later, treated with 16 mM HU for additional 62 h (siRNA→HU). All cells were fixed and stained with anti-centrobin and γ-tubulin antibodies. Data presented are the percentages of cells with more than two centrosomes (average and standard deviation from three independent experiments with 300 cells counted in every experiment). FIG. 6G is a set of photographs of representative U2OS cells transfected with control or centrobin siRNA.

FIG. 7 is a series of graphs and photographs showing centrobin depletion leads to impaired cytokinesis. FIG. 7A is a bar graph quantitating the percentage of cells with multiple nuclei transfected with control or centrobin siRNA. HeLa cells were transfected with scrambled siRNA or centrobin siRNA and, 72 h later, stained with anti-centrobin and DAPI (blue). The percentage of cells with two or more nuclei was enumerated. Data presented are averages and standard deviations from three independent experiments with 300 cells counted in every experiment. FIG. 7B is a set of photographs showing representative HeLa cells with two or four nuclei. FIG. 7C is a chart showing that centrobin depletion led to impaired cytokinesis. HeLa cells were transfected with scrambled siRNA or centrobin siRNA. Eight hours after transfection, time-lapse phase contrast microscopy was initiated and continued for 48 h. All the cells that underwent cytokinesis were followed to determine the fate and duration of cell division (from cell roundup till two daughter cells separate). The data presented were compiled from two experiments. The durations of cell division for the 23 control cells are 50, 55, 65, 65, 70, 75, 80, 80, 80, 85, 90, 100, 100, 105, 110, 140, 160, 165, 170, 180, 200, 220, 450 minutes. The durations of cell division for the 11 centrobin-siRNA transfected-cells are 85, 115, 135, 135, 220, 270, 335, 600, 640, 645, 665 minutes. Four centrobin-siRNA transfected-cells exited mitosis with finishing cytokinesis at 175, 190, 210, 260 min. FIG. 7D is a series of photographs of time-lapse phase contrast microscopy of representative HeLa cells that underwent mitosis, which were transfected with centrobin siRNA or were transfected with control siRNA. The centrobin siRNA transfected cell was in mitosis for more than 260 min and exited without finishing mitosis. The control siRNA transfected cells took less than 120 min to finish mitosis.

FIG. 8 is a diagram showing the inhibition of centrosome duplication by centrobin depletion that will induce cell death of cancer cells and growth arrest of normal cells.

FIG. 9A is a bar graph of centrobin RNAi induced cell death in HeLa cells and FIG. 9B is a bar graph of centrobin RNAi induced cell death in 76NTert cells. Centrobin depletion induced cell death in HeLa cells and 76NTert cells. Cells plated in 24 wells were transfected with centrobin RNAi or scrambled RNAi. The percentage of dead cells were determined by trypan blue exclusion assay at the indicated time points after transfection.

FIG. 10 is a photograph of the centrin and centrobin stained using DAPI, which show that centrobin depletion in normal cells induced inhibition of centrosome duplication and led to cells with split and unduplicated centrioles. The untransformed cells, 76NTert cells were transfected with centrobin RNAi.

### DETAILED DESCRIPTION OF THE INVENTION

In mammalian cells, the centrosome is comprised of a pair of centrioles and amorphous pericentriolar material. Centrosomes play a pivotal role in orchestrating the formation of the bipolar spindle during mitosis. Recent studies have also linked the centrosome activity to cytokinesis and the activation of DNA replication. The centrosome duplicates once and only once during each cell cycle; the duplication starts at the G1/S transition and is completed by G2. Normal centrosome duplication is a key requirement for correct segregation of chromosomes during cell division, and the centrosome duplication cycle is tightly coupled to the cell division cycle. Uncoupling of the centrosome duplication cycle and the cell division cycle leads to more than two centrosomes per cell, i.e. centrosome amplification, a phenotype frequently observed in both in vitro cultured tumor cells and a variety of tumors at different stages.

Centrosomes duplicate once per cell cycle just like chromosomes do. While the mechanism of DNA duplication was well established after Watson and Crick discovered the structure of DNA, how centrosomes duplicate remained to be determined. From electron microscopy observation, normal somatic cells in G0 and G1 phase were found to contain one centrosome with two centrioles. One of the two centrioles is generated two cycles prior and has appendages, is usually referred to as mother centriole. The other centriole is generated in previous cell cycle and has no appendages, is also referred to as daughter centriole. The daughter centriole is orthogonal to the mother centriole. When the cells are at the G1/S transition, the centriole pair split and mother centriole and daughter centriole become parallel to each other. The new centrioles start to grow from the side of two parallel original centrioles. The newly synthesized centrioles elongate to the same length of the original centrioles at the end of S phase or the beginning of G2/M phase. At the beginning of M phase, the amount of pericentriolar material accumulates significantly around the centrioles and then two centrosomes separate to form the two spindle poles. Similar to DNA replication, centrosome duplication can also be considered as semiconservative duplication because the newly synthesized centrosome has one old centriole and one new centriole.

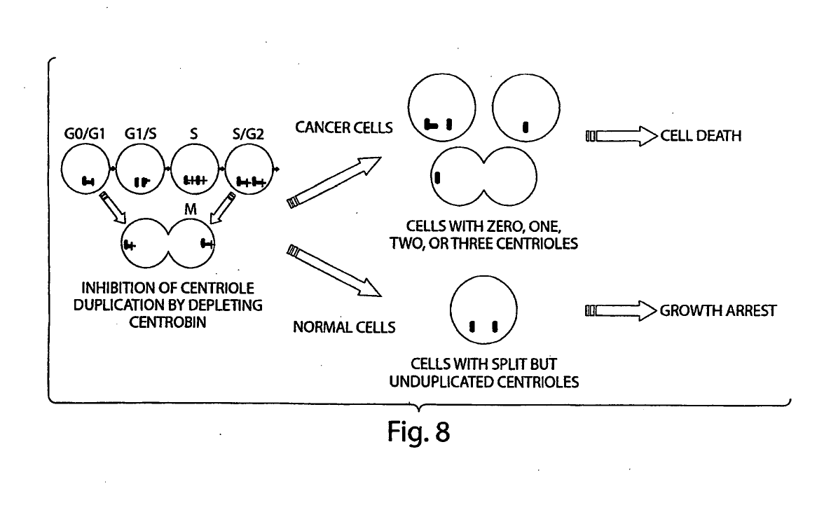

For the last sixty years, chemotherapeutic agents used for cancer were mainly targeting DNA duplication. As described herein, centrosome duplication was found to be an effective target for cancer therapy. Targeting centrosome duplication has many advantages over targeting DNA duplication. Centrosome duplication is always coupled to DNA duplication in normal cells, but is very frequently uncoupled from DNA duplication in cancer cells. Therefore, when centrosome duplication is inhibited, the normal cells are arrested in G1 phase while the cancer cells continue to divide to produce cells with one, three or no centrioles. The abnormal number of centrioles leads to abnormal cell divisions and eventually cancer cell death (see FIG. 8). The normal cells are still functional while there are in G1 arrest and should resume their proliferation when centrosome duplication inhibitors are removed due to the decay of the inhibitors. Additionally, inhibition of centrosome duplication does not cause heritable DNA mutations that can be transmitted to the progenies, which is a drawback of the current chemotherapeutic agents that target DNA duplication. Inhibition of centrosome duplication does not induce cellular transformation as many current chemotherapeutic agents do.

Centrobin was identified as a BRCA2 binding protein, and it asymmetrically localizes to the daughter centriole and is required for the duplication of centriole. Depletion of centrobin leads to inhibition of centriole duplication, hence the centrosome duplication. Furthermore, centrobin depletion in cancer cells led to extensive cell death, while depletion of centrobin in normal cells led to growth arrest of the normal cells, as described above. Targeting centrobin inhibits centrosome duplication and kills cancer cells specifically while sparing the normal cells. The method to target centrobin includes depletion of centrobin by RNAi or modified anti-sense sequences, or inhibition of centrobin function by other methods such as inhibiting its modifications or promoting its degradation.

The invention is based in part on the discovery of a novel centrosomal protein. The protein was identified in a yeast two hybrid system using a C-terminal fragment of BRCA2 as bait. The protein is referred to as centrobin (Centrosomal BRCA2 interacting protein). Centrobin interacts with BRCA2 both in vitro and in vivo with high affinity.

Molecular cloning of the full length centrobin identified two transcripts. A major transcript of 3718 nucleotides (SEQ ID NO:1; Table 1. The open reading frame is shown in bold text.) encoding a 903 amino acid polypeptide (SEQ ID NO:2; Table 2) and a minor transcript of 3784 nucleotides (SEQ ID NO: 3; Table 3) encoding a 925 amino acid polypeptide (SEQ ID NO:4; Table 4). Sequence comparison of the two transcripts revealed that the minor transcript contained a 63 bp in-frame insertion after nucleotide 3433 of the major transcript, shown in bold in SEQ ID NO: 3, which encodes a 21 amino acid sequence (SEQ ID NO: 6) shown in bold in SEQ ID NO: 4. The 3718 bp transcript is referred to as centrobin α and the 3,784 bp transcript is referred to as centrobin β.

TABLE 1
Centrobin α Nucleic Acid Sequence (SEQ ID NO: 1)

gtggtctcgcgggcgggtgacgtcatcgaggcgagagaggcggagccgtggacagtgggcgggaggctgccaacggtttt
gagcgtagggggaggcgtgagagggggatctcaggggaggaggtcaatcgcttgccccccactttggcaaattggggact
gaggactggaagggtggagagtaggcggaaccaggtggtcgtcggggcagaggatctcgggctaggcttgagggcggcgt
gcttcttagggacgacttagggcgtgactgagggttcacaaggtttcttttggggtggtcgggagggagagattctaggg
aacaaggaagctcgctatggctttcttgccaggaggggtcgaagggaaagtacaagggagctgaccctgggtagaacggg
tgaagggatgggggagcgtgaggttccgccctctcttgagactggaaccaattgagggactagtagggcagggggacaga
aattgggctcctagtggatttgggtccgtttccgttgggacgttttgggtgtgagaacttaagagctcagttgaccgggg
atagcctgtgccggagttgatctgcagcttccagcactcgtagtcgggaagaggagcttcagcagcgctgttgtcccaca
gtaggtcttctgtccgcacccgctctgcgctgcaccctcttaacgctgttcccaggagctggggaaagggatgcttttgc
ccactcccatggcccctggaactggtggaaacctttcctctaaccagaaagcctcgatatccttaattcaccaaggatcc
ttggcgtggagtcttcctcccttctcccaagtctttctccgtgaacttttcctcctggactttgctaaagcagaacctcc
cagctctttgctgtctccggttgtctcttccctgtattcatggcaacatcagctgacagccccagttcacccctcggggc
ggaggatctcctgagtgattcatcagaaccccctgggctcaaccaagtgtcgtctgaagtgacctcccagctctatgctt
ctttgcgcctcagccggcaggcggaggccacggcccgagcccagctgtatttaccctccacctccccgcctcatgaaggg
ttagacggcttcgcccaagaattgagtcgaagcttgtcagtcggattggaaaagaacttgaagaaaaaggatggttctaa
gcatatctttgagatggaaagtgttcggggtcagctccagaccatgctccaaacctcacgtgatacagcctatcgggatc
ctctcattcctggcgctggctcagagagacgggaagaggactcctttgacagtgatagcacagccaccttgctcaacacc
cggcccctgcaagacttgtctccatctagctcagcccaagccctggaggagctgtttccccgctacaccagccttcggcc
agggcctccactcaatcccccagattttcaggggctgagagatgcattggattcagagcatacccgccgcaagcattgtg
agcgccatattcagagcctgcagacccgagtgttagagctacagcaacaattagccgtggctgtggctgccgaccgcaag
aaagataccatgattgaacaactggacaagaccctggcccgtgtggtggagggctggaaccggcatgaggctgagcggac
agaggttctcaggggacttcaagaggaacaccaggcagcagagctcaccagaagcaagcagcaggagacagtaacccgcc
tggaacaaagcctttctgaggccatggaggccctgaatcgtgagcaggaaagtgccagactgcagcaacgggaaagagag
acactggaggaggaaaggcaagctctgactctgaggttggaggcagaacagcagcggtgctgtgtcctgcaggaagagcg
ggatgcagctcgggctgggcaactgagtgagcatcgagagttggagactcttcgggctgccctagaagaagaacggcaga
cctgggcccagcaagagcaccagcttaaggaacactaccaggcgctgcaggaggagagccaggctcagctggaaagggag
aaggagaagagccagagggaagcccaggccgcctgggagacccagcaccagttggcattggtgcagtctgaggtgcggcg
gctggaaggagagctggatacagctcggagagagagagatgccctgcagctggaaatgagcttggtgcaggcccggtatg
aaagccagcggatccagctggagtcggagctggctgtgcagctggagcagcgggtgacagagcggctggcgcaggctcag
gagagcagcctacggcaagcagcctccctcagggaacatcacaggaagcagctgcaggacctgagtggacagcaccagca
ggagctggccagtcagctagctcagttcaaggtggaaatggcagaacgagaggaacggcaacagcaggtggctgaggact
acgagctcagactggcccgggagcaagcgcgagtgtgcgaactgcagagtgggaaccagcagctggaggagcagcgggtg
gagctggtggaaagactgcaggccatgctgcaggcccactgggatgaggccaaccagctgctcagcaccactctcccgcc
gcccaaccctccagctcctcctgctggaccctccagccccgggcctcaggagcccgagaaggaggagaggagggtctgga
ctatgcctcccatggccgtggccctgaagcctgtattgcagcagagccgggaagcaagggacgagctacctggagcgcct
cctgttctttgcagttcctcctcagatcttagcctcctgttgggcccctcttttcagagccagcattctttccagcccct
ggagcccaaaccagacctcacttcatccacagctggggccttctctgcacttggggccttccatcccgatcatagggcag
aaaggccattccctgaggaagatcctggacctgacggggagggcctcctaaagcaagggctgccgcctgctcagctggag
ggcctcaagaattttttgcaccagttgctggagacagtgccccagaacaatgagaacccttctgtcgacctgttgccccc
taagtctggtcctctgactgtcccatcttgggaggaagcccctcaagtgccacgtattccaccgcctgtccacaaaacca
aagttcccttagccatggcatccagtcttttccgggtccctgagcctccctcctcccattcacaaggcagtggtcccagc
agtggttccccagagagaggtggagatgggcttacattcccaaggcagctgatggaggtgtctcaactgttgcgactcta
ccaggctcggggctggggggctctgcctgctgaggatctcctgctctacctgaagaggctggaacacagcgggactgatg
gccgaggggataatgtccccagaaggaacacagactcccgcttgggtgagatcccccggaaagagattccctcccaggct
gtccctcgccgccttgctacagcccccaagactgaaaaacctcccgcacggaagaaaagtgggcaccctgccccgagtag
catgaggagccgggggggagtctggagatgagcccccctaccctctctcctctttgttctctcattgttgttattttaat
aaatgctcattagtctgcaaaaaaaaaaaaaaaaaaaa

TABLE 2
Centrobin α Amino Acid Sequence (SEQ ID NO: 2)

MATSADSPSSPLGAEDLLSDSSEPPGLNQVSSEVTSQLYASLRLSRQAEATARAQLYLPSTSPPHEGLDGFAQE
LSRSLSVGLEKNLKKKDGSKHIFEMESVRGQLQTMLQTSRDTAYRDPLIPGAGSERREEDSFDSDSTATLLNTR
PLQDLSPSSSAQALEELFPRYTSLRPGPPLNPPDFQGLRDALDSEHTRRKHCERHIQSLQTRVLELQQQLAVAV
AADRKKDTMIEQLDKTLARVVEGWNRHEAERTEVLRGLQEEHQAAELTRSKQQETVTRLEQSLSEAMEALNREQ
ESARLQQRERETLEEERQALTLRLEAEQQRCCVLQEERDAARAGQLSEHRELETLRAALEEERQTWAQQEHQLK
EHYQALQEESQAQLEREKEKSQREAQAAWETQHQLALVQSEVRRLEGELDTARRERDALQLEMSLVQARYESQR
IQLESELAVQLEQRVTERLAQAQESSLRQAASLREHHRKQLQDLSGQHQQELASQLAQFKVEMAEREERQQQVA
EDYELRLAREQARVCELQSGNQQLEEQRVELVERLQAMLQAHWDEANQLLSTTLPPPNPPAPPAGPSSPGPQEP
EKEERRVWTMPPMAVALKPVLQQSREARDELPGAPPVLCSSSSDLSLLLGPSFQSQHSFQPLEPKPDLTSSTAG
AFSALGAFHPDHRAERPFPEEDPGPDGEGLLKQGLPPAQLEGLKNFLHQLLETVPQNNENPSVDLLPPKSGPLT
VPSWEEAPQVPRIPPPVHKTKVPLAMASSLFRVPEPPSSHSQGSGPSSGSPERGGDGLTFPRQLMEVSQLLRLY
QARGWGALPAEDLLLYLKRLEHSGTDGRGDNVPRRNTDSRLGEIPRKEIPSQAVPRRLATAPKTEKPPARKKSG
HPAPSSMRSRGGVWR

TABLE 3
Centrobin β Nucleic Acid Sequence (SEQ ID NO: 3)

gtggtctcgcgggcgggtgacgtcatcgaggcgagagaggcggagccgtggacagtgggcgggaggctgccaacggtttt
gagcgtagggggaggcgtgagagggggatctcaggggaggaggtcaatcgcttgccccccactttggcaaattggggact
gaggactggaagggtggagagtaggcggaaccaggtggtcgtcggggcagaggatctcgggctaggcttgagggcggcgt
gcttcttagggacgacttagggcgtgactgagggttcacaaggtttcttttggggtggtcgggagggagagattctaggg
aacaaggaagctcgctatggctttcttgccaggaggggtcgaagggaaagtacaagggagctgaccctgggtagaacggg
tgaagggatgggggagcgtgaggttccgccctctcttgagactggaaccaattgagggactagtagggcagggggacaga
aattgggctcctagtggatttgggtccgtttccgttgggacgttttgggtgtgagaacttaagagctcagttgaccgggg
atagcctgtgccggagttgatctgcagcttccagcactcgtagtcgggaagaggagcttcagcagcgctgttgtcccaca
gtaggtcttctgtccgcacccgctctgcgctgcaccctcttaacgctgttcccaggagctggggaaagggatgcttttgc
ccactcccatggcccctggaactggtggaaacctttcctctaaccagaaagcctcgatatccttaattcaccaaggatcc
ttggcgtggagtcttcctcccttctcccaagtctttctccgtgaacttttcctcctggactttgctaaagcagaacctcc
cagctctttgctgtctccggttgtctcttccctgtattcatggcaacatcagctgacagccccagttcacccctcggggc
ggaggatctcctgagtgattcatcagaaccccctgggctcaaccaagtgtcgtctgaagtgacctcccagctctatgctt
ctttgcgcctcagccggcaggcggaggccacggcccgagcccagctgtatttaccctccacctccccgcctcatgaaggg
ttagacggcttcgcccaagaattgagtcgaagcttgtcagtcggattggaaaagaacttgaagaaaaaggatggttctaa
gcatatctttgagatggaaagtgttcggggtcagctccagaccatgctccaaacctcacgtgatacagcctatcgggatc
ctctcattcctggcgctggctcagagagacgggaagaggactcctttgacagtgatagcacagccaccttgctcaacacc
cggcccctgcaagacttgtctccatctagctcagcccaagccctggaggagctgtttccccgctacaccagccttcggcc
agggcctccactcaatcccccagattttcaggggctgagagatgcattggattcagagcatacccgccgcaagcattgtg
agcgccatattcagagcctgcagacccgagtgttagagctacagcaacaattagccgtggctgtggctgccgaccgcaag
aaagataccatgattgaacaactggacaagaccctggcccgtgtggtggagggctggaaccggcatgaggctgagcggac
agaggttctcaggggacttcaagaggaacaccaggcagcagagctcaccagaagcaagcagcaggagacagtaacccgcc
tggaacaaagcctttctgaggccatggaggccctgaatcgtgagcaggaaagtgccagactgcagcaacgggaaagagag
acactggaggaggaaaggcaagctctgactctgaggttggaggcagaacagcagcggtgctgtgtcctgcaggaagagcg
ggatgcagctcgggctgggcaactgagtgagcatcgagagttggagactcttcgggctgccctagaagaagaacggcaga
cctgggcccagcaagagcaccagcttaaggaacactaccaggcgctgcaggaggagagccaggctcagctggaaagggag
aaggagaagagccagagggaagcccaggccgcctgggagacccagcaccagttggcattggtgcagtctgaggtgcggcg
gctggaaggagagctggatacagctcggagagagagagatgccctgcagctggaaatgagcttggtgcaggcccggtatg
aaagccagcggatccagctggagtcggagctggctgtgcagctggagcagcgggtgacagagcggctggcgcaggctcag
gagagcagcctacggcaagcagcctccctcagggaacatcacaggaagcagctgcaggacctgagtggacagcaccagca
ggagctggccagtcagctagctcagttcaaggtggaaatggcagaacgagaggaacggcaacagcaggtggctgaggact
acgagctcagactggcccgggagcaagcgcgagtgtgcgaactgcagagtgggaaccagcagctggaggagcagcgggtg
gagctggtggaaagactgcaggccatgctgcaggcccactgggatgaggccaaccagctgctcagcaccactctcccgcc
gcccaaccctccagctcctcctgctggaccctccagccccgggcctcaggagcccgagaaggaggagaggagggtctgga
ctatgcctcccatggccgtggccctgaagcctgtattgcagcagagccgggaagcaagggacgagctacctggagcgcct
cctgttctttgcagttcctcctcagatcttagcctcctgttgggcccctcttttcagagccagcattctttccagcccct
ggagcccaaaccagacctcacttcatccacagctggggccttctctgcacttggggccttccatcccgatcatagggcag
aaaggccattccctgaggaagatcctggacctgacggggagggcctcctaaagcaagggctgccgcctgctcagctggag
ggcctcaagaattttttgcaccagttgctggagacagtgccccagaacaatgagaacccttctgtcgacctgttgccccc
taagtctggtcctctgactgtcccatcttgggaggaagcccctcaagtgccacgtattccaccgcctgtccacaaaacca
aagttcccttagccatggcatccagtcttttccgggtccctgagcctccctcctcccattcacaaggcagtggtcccagc
agtggttccccagagagaggtggagatgggcttacattcccaaggcagctgatggaggtgtctcaactgttgcgactcta
ccaggctcggggctggggggctctgcctgctgaggatctcctgctctacctgaagaggctggaacacagcgggtacaagc
ctgggaggaaggaggaaggattctccgggtggaagctggattatggggagtggagtgggactgatggccgaggggataat
gtccccagaaggaacacagactcccgcttgggtgagatcccccggaaagagattccctcccaggctgtccctcgccgcct
tgctacagcccccaagactgaaaaacctcccgcacggaagaaaagtgggcaccctgccccgagtagcatgaggagccggg
ggggagtctggagatgagcccccctaccctctctcctctttgttctctcattgttgttattttaataaatgctcattagt
ctgcaaaaaaaaaaaaaaaaaaaa

TABLE 4
Centrobin β Amino Acid Sequence (SEQ ID NO: 4)

MATSADSPSSPLGAEDLLSDSSEPPGLNQVSSEVTSQLYASLRLSRQAEATARAQLYLPSTSPPHEGLDGFAQELSRSLS
VGLEKNLKKKDGSKHIFEMESVRGQLQTMLQTSRDTAYRDPLIPGAGSERREEDSFDSDSTATLLNTRPLQDLSPSSSAQ
ALEELFPRYTSLRPGPPLNPPDFQGLRDALDSEHTRRKHCERHIQSLQTRVLELQQQLAVAVAADRKKDTMIEQLDKTLA
RVVEGWNRHEAERTEVLRGLQEEHQAAELTRSKQQETVTRLEQSLSEAMEALNREQESARLQQRERETLEEERQALTLRL
EAEQQRCCVLQEERDAARAGQLSEHRELETLRAALEEERQTWAQQEHQLKEHYQALQEESQAQLEREKEKSQREAQAAWE
TQHQLALVQSEVRRLEGELDTARRERDALQLEMSLVQARYESQRIQLESELAVQLEQRVTERLAQAQESSLRQAASLREH
HRKQLQDLSGQHQQELASQLAQFKVEMAEREERQQQVAEDYELRLAREQARVCELQSGNQQLEEQRVELVERLQAMLQAH
WDEANQLLSTTLPPPNPPAPPAGPSSPGPQEPEKEERRVWTMPPMAVALKPVLQQSREARDELPGAPPVLCSSSSDLSLL
LGPSFQSQHSFQPLEPKPDLTSSTAGAFSALGAFHPDHRAERPFPEEDPGPDGEGLLKQGLPPAQLEGLKNFLHQLLETV
PQNNENPSVDLLPPKSGPLTVPSWEEAPQVPRIPPPVHKTKVPLAMASSLFRVPEPPSSHSQGSGPSSGSPERGGDGLTF
PRQLMEVSQLLRLYQARGWGALPAEDLLLYLKRLEHSGYKPGRKEEGFSGWKLDYGEWSGTDGRGDNVPRRNTDSRLGEI
PKKEOPSQAVPRRLATAPKTEKPPARKKSGHPAPSSMRSRGGVWR

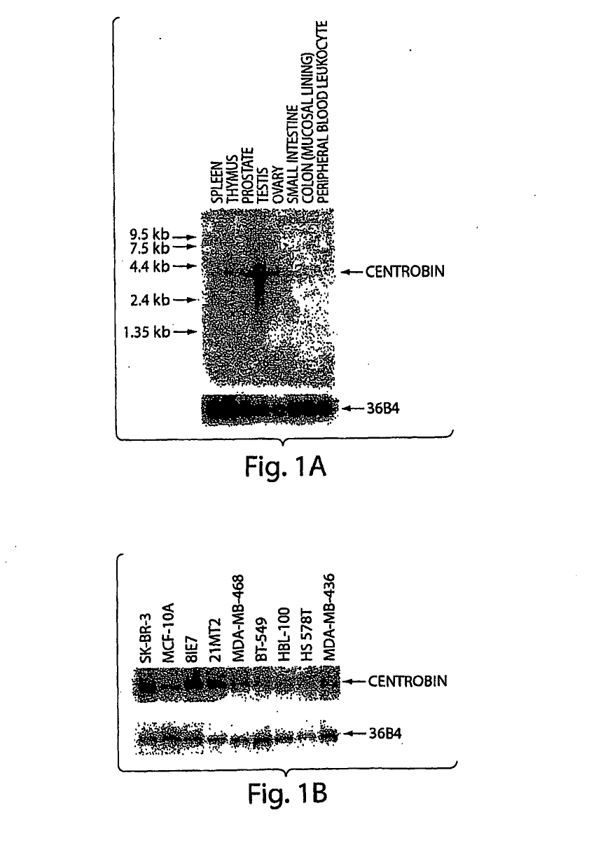

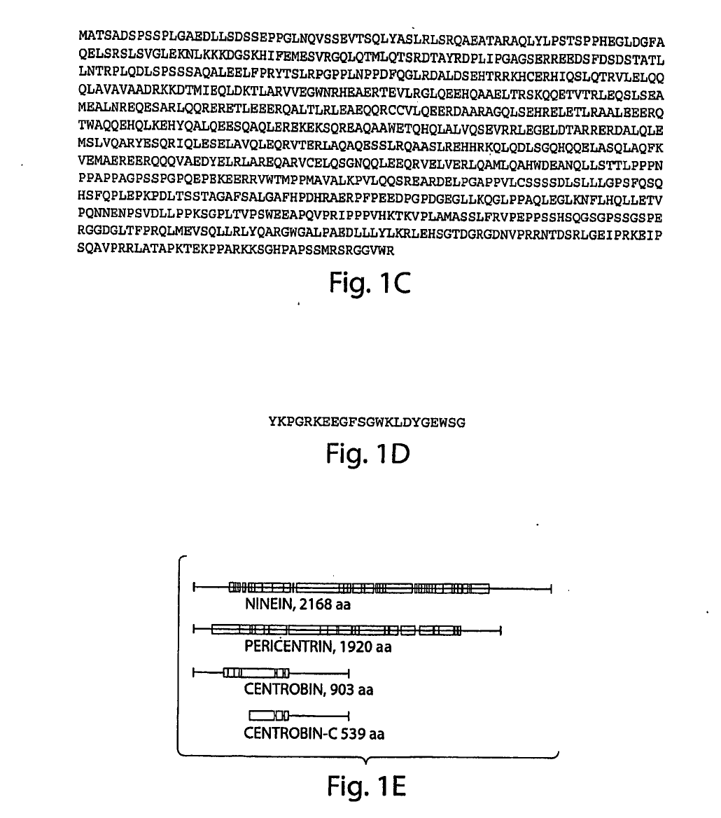

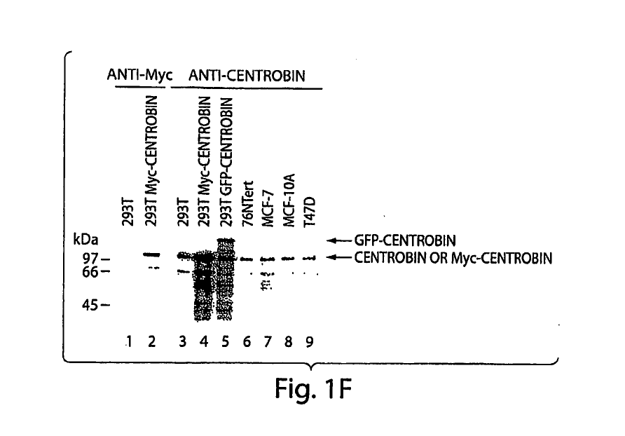

Sequence analysis showed that centrobin shares homology to various coil-coiled proteins, including two centrosomal proteins, pericentrin and ninein. Centrobin shows 25% identity and 46% similarity with pericentrin within a region of 323 amino acids. Pericentrin is an integral component of the pericentriolar matrix of the centrosomes that has been shown to regulate centrosome assembly and organization. In addition, centrobin shows 25% identity and 43% similarity with ninein within a region of 376 amino acids. Ninein is a component of the centrosome matrix and associates with centrosomes at all stages of the cell cycle and is a potential microtubule-anchoring protein. An alpha helical domain is located at amino acids 189-619 of centrobin α and centrobin β. Similar to pericentrin and ninein, centrobin is predicted to have a coiled-coil region in the middle and non-coiled regions at both the C-terminal end and the N-terminal end (See, FIG. 1E).

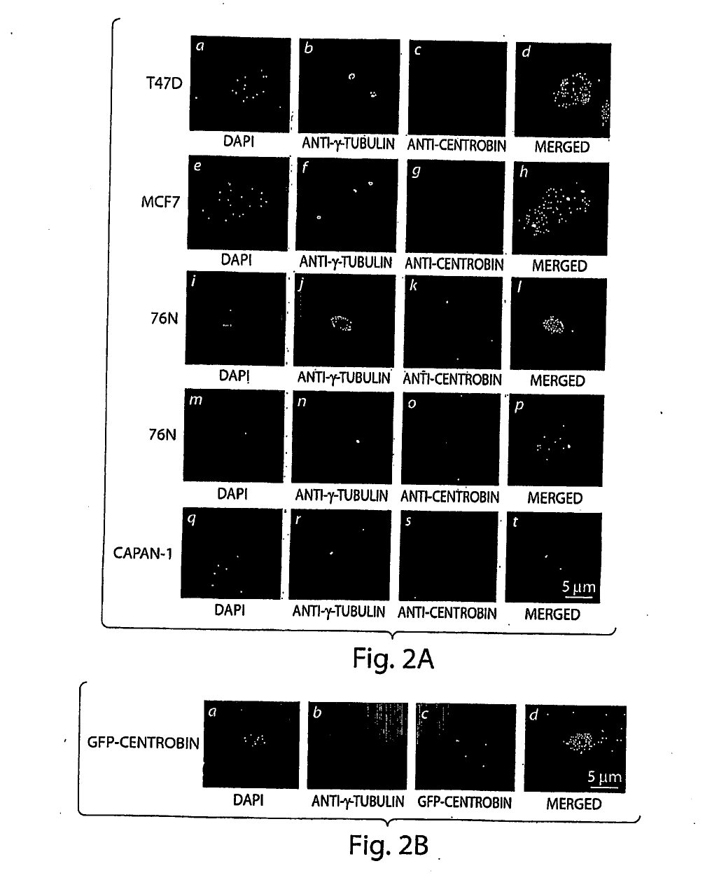

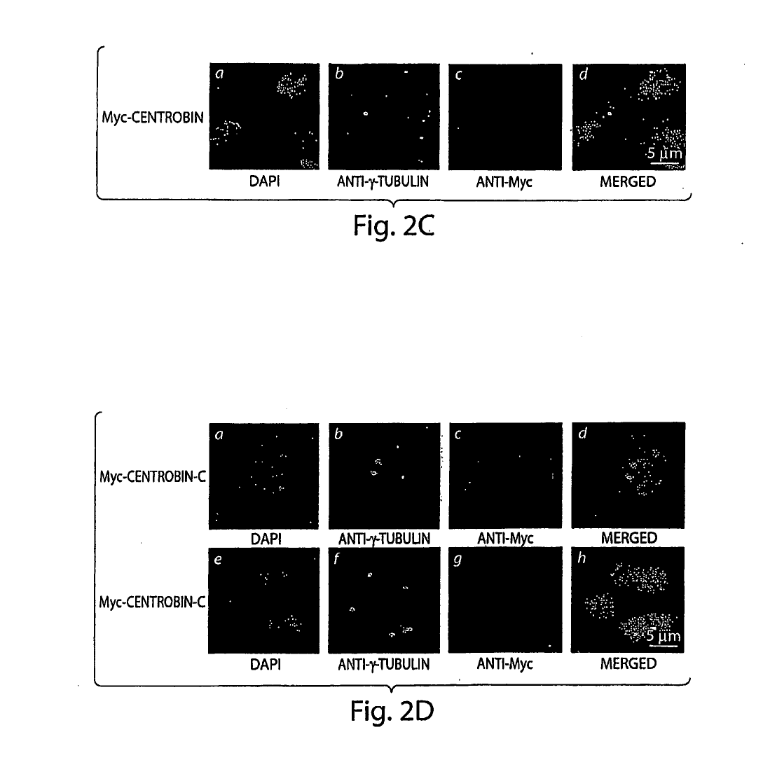

Immunofluorescence studies using an anti-centrobin antibody demonstrated that centrobin is localized to the centrosomes during both interphase and the mitotic phase. (See, FIG. 2.) This localization is independent of BRAC2 interaction. Depolymerizing the microtubules by nocodazole treatment did not affect the centrosomal localization of centrobin, indicating that centrobin is an integral component of the centrosomes. Using a myc-centrobin construct encoding the C-terminal 539 amino acids (underlined in Table 2; referred to herein as centrobin-C) it was determined that this C-terminal fragment is sufficient to confer on centrobin the ability to localize to the centrosomes.

When the full-length centrobin is expressed at high level in cells, it forms rod-like structures in the cytoplasm of the transfected cells, which is similar to other centrosomal proteins such as the TACC family proteins. These rod-like structures contain both α-tubulin and γ-tubulins. In addition, it was shown that BRACA2 is also incorporated into these rod-like structures. Overexpressed centrobin-C but not endogenous centrobin localizes to the microtubules. The data indicate that centrobin is able to bind to α-tubulin and γ-tubulin either directly or indirectly. As both α-tubulin and γ-tubulin are integral components of the centrosomes, centrobin appears to localize to the centrosomes via its interaction with α- or γ-tubulins.

BRCA2 interacts with centrobin both in vitro and in vivo. The centrobin binding domain on BRCA2 at its C-terminal region (amino acids 2393-2952, referred to herein as BRCA2-C3). (See Genbank Accession Number P51587.) The BRCA2 binding domain on centrobin localizes to the C-terminal region of centrobin. Specifically, the BRCA2 binding domain of centrobin α includes amino acids 365-904, and the BRCA2 binding domain of centrobin β includes amino acids 365-925. Overexpression of these binding fragments disrupts the interaction of endogenous BRCA2 and centrobin in vivo and demonstrates that interaction of BRCA2 and centrobin plays an important role in normal centrosome duplication.

### Centrobin Nucleic Acids

In one aspect of the invention provides isolated nucleic acid molecules that encode centrobin proteins or biologically active portions thereof. Also included are nucleic acid fragments for use as hybridization probes to identify centrobin-encoding nucleic acids or for use as polymerase chain reaction (PCR) primers for the amplification or mutation of centrobin nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule is single-stranded or double-stranded.

“Probes” refer to nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes are single- or double-stranded and designed to have specificity in PCR, membrane-based hybridization technologies, or ELISA-like technologies. The probe further contains a label group attached thereto, e.g. the label group is a radioisotope, a fluorescent compound, an enzyme, or an enzyme co-factor.

An “isolated” nucleic acid molecule is one that is separated from other nucleic acid molecules that are present in the natural source of the nucleic acid. Examples of isolated nucleic acid molecules include, for example, recombinant DNA molecules contained in a vector, recombinant DNA molecules maintained in a heterologous host cell, partially or substantially purified nucleic acid molecules, and synthetic DNA or RNA molecules. An “isolated” nucleic acid is free of sequences which naturally flank the nucleic acid (i.e., sequences located at the 5′ and 3′ ends of the nucleic acid) in the genomic DNA of the organism from which the nucleic acid is derived. For example, the isolated centrobin nucleic acid molecule contains less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, is substantially free of other cellular material or culture medium when produced by recombinant techniques, or of chemical precursors or other chemicals when chemically synthesized.

As used herein, the term “oligonucleotide” refers to a series of linked nucleotide residues, which oligonucleotide has a sufficient number of nucleotide bases to be used in a PCR reaction. A short oligonucleotide sequence is based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 20 nt, 50 nt, or 100 nt in length, preferably about 15 nt to 30 nt in length.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, Van der Waals, hydrophobic interactions, etc. A physical interaction is either direct or indirect. Indirect interactions are through or due to the effects of another polypeptide or compound. Direct binding refers to interactions that do not take place through, or due to, the effect of another polypeptide or compound.

Fragments are defined as sequences of at least 6 (contiguous) nucleic acids, a length sufficient to allow for specific hybridization and are less than a full length naturally-occurring gene sequence. Fragments are derived from any contiguous portion of a nucleic acid or amino acid sequence of choice. For example, an exemplary isolated nucleic acid molecule of the invention, comprises contiguous nucleotides encoding amino acid residues 371-903 of SEQ ID NO:2.

Derivatives are nucleic acid sequences that contain a modified nucleotide or nucleotide substitution compared to a reference sequence. Analogs are nucleic acid sequences that have a structure similar to, but not identical to, the native compound but differs from it in respect to certain components or side chains. Homologs are nucleic acid sequences of a particular gene that are derived from different species.

Derivatives, homologs and analogs are full length or differ in length compared to a reference sequence. Derivatives or analogs of the nucleic acids or proteins of the invention include, but are not limited to, molecules containing regions that are homologous to the nucleic acids or proteins of the invention by at least about 30%, 50%, 70%, 80%, 85%, 90%, 95%, 97%, 98% or 99% identity over a nucleic acid or amino acid sequence of identical size or when compared to an aligned sequence in which the alignment is done by a computer homology program known in the art.

For example, a homologous nucleotide sequences encode isoforms of a centrobin polypeptide; in some embodiments, an isoform is a result of alternative splicing of RNA. Alternatively, isoforms can be encoded by different genes. Homologous nucleotide sequences include nucleotide sequences encoding for a centrobin polypeptide of species other than humans, including, but not limited to, mammals, and thus can include, e.g., mouse, rat, rabbit, dog, cat, cow, horse, and other organisms. Homologous nucleotide sequences also include, for example, naturally occurring allelic variations and mutations of the nucleotide sequences set forth herein.

As used herein, the term “hybridizes under stringent conditions” is intended to describe conditions for hybridization and washing under which nucleotide sequences at least 60% identical to each other typically remain hybridized to each other.

As used herein, the phrase “stringent hybridization conditions” refers to conditions under which a probe, primer or oligonucleotide will specifically hybridize to its target sequence. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Stringent conditions include those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C.

### Centrobin Polypeptides

The invention provides isolated centrobin proteins, and biologically active portions thereof, or derivatives, fragments, analogs or homologs thereof. By biologically active portion is meant that the peptide has an activity associated with full-length centrobin, such as binding to BRCA-2 or centrosomal localization. Native centrobin proteins are isolated from cells or tissue sources by an appropriate purification scheme using standard protein purification techniques. Alternatively, centrobin proteins are produced by recombinant DNA techniques or synthesized chemically using standard peptide synthesis techniques.

Homologous amino acid sequences include amino acid sequences of centrobin polypeptides of species other than humans, including, but not limited to, mammals, and thus can include, e.g., mouse, rat, rabbit, dog, cat, cow, horse, and other organisms.

An “isolated” or “purified” protein or biologically active portion thereof is substantially free of cellular material or other contaminating proteins from the cell or tissue source from which the centrobin protein is derived, or substantially free from chemical precursors or other chemicals when chemically synthesized. The language “substantially free of cellular material” includes preparations of centrobin protein, in which the protein is separated from cellular components of the cells from which it is isolated or recombinantly produced. “Substantially free of cellular material” includes preparations of centrobin protein having less than about 30% (by dry weight) of non-centrobin protein (also referred to herein as a “contaminating protein”), more preferably less than about 20% of non-centrobin protein, still more preferably less than about 10% of non-centrobin protein, and most preferably less than about 5%, 2%, 1% non-centrobin protein. When the centrobin protein or biologically active portion thereof is recombinantly produced, it is also preferably substantially free of culture medium, i.e., culture medium represents less than about 20%, more preferably less than about 10%, and most preferably less than about 5%, 2%, 1% of the volume of the protein preparation.

The language “substantially free of chemical precursors or other chemicals” includes preparations of centrobin protein in which the protein is separated from chemical precursors or other chemicals that are involved in the synthesis of the protein. “Substantially free of chemical precursors or other chemicals” includes preparations of centrobin protein having less than about 30% (by dry weight) of chemical precursors or non-centrobin chemicals, more preferably less than about 20% chemical precursors or non-centrobin chemicals, still more preferably less than about 10% chemical precursors or non-centrobin chemicals, and most preferably less than about 5%, 2%, 1% chemical precursors or non-centrobin chemicals.

Biologically active portions of a centrobin protein include peptides comprising amino acid sequences sufficiently homologous to or derived from the amino acid sequence of the centrobin protein, e.g., the amino acid sequence shown in SEQ ID NO: 2 or 4 that include fewer amino acids than the full length centrobin proteins, and exhibit at least one activity of a centrobin protein. A biologically active portion of a centrobin protein is a polypeptide which is, 10, 25, 50, 100, 250, 500 or more amino acids in length but less than the full-length naturally-occurring protein. For example, a biologically active portion of centrobin includes amino acids 371-903 of SEQ ID NO:2.

The centrobin protein has an amino acid sequence shown in SEQ ID NO: 2 or 4. Alternatively, the centrobin protein is substantially homologous to SEQ ID NO: 2 or 4, and retains the functional activity of the protein of SEQ ID NO: 2 or 4, yet differs in amino acid sequence due to natural allelic variation or mutagenesis.

A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the centrobin protein is replaced with another amino acid residue from the same side chain family. Alternatively, mutations can be introduced randomly along all or part of a centrobin coding sequence, such as by saturation mutagenesis, and the resultant mutants can be screened for centrobin biological activity to identify mutants that retain activity.

The invention also provides centrobin chimeric or fusion proteins. As used herein, a centrobin “chimeric protein” or “fusion protein” comprises a centrobin polypeptide operatively linked to a non-centrobin polypeptide. A “centrobin polypeptide” refers to a polypeptide having an amino acid sequence corresponding to centrobin, whereas a “non-centrobin polypeptide” refers to a polypeptide having an amino acid sequence corresponding to a protein that is different from the centrobin protein and that is derived from the same or a different organism. Within a centrobin fusion protein the centrobin polypeptide can contain all or a portion of a centrobin. A centrobin fusion protein comprises at least one biologically active portion of a centrobin protein. The non-centrobin polypeptide is fused to the N-terminus or C-terminus of the centrobin polypeptide. Alternatively, the non-centrobin polypeptide is inserted within the centrobin polypeptide or replaces a portion of the centrobin polypeptide.

A centrobin chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide).

### Anti-Centrobin Antibodies

The invention encompasses antibodies and antibody fragments, such as F_{ab} or (F_{ab})₂, that bind immunospecifically to any of the polypeptides of the invention. An isolated centrobin protein, or a portion or fragment thereof, is used as an immunogen to generate antibodies that bind to a centrobin polypeptide using standard techniques for polyclonal and monoclonal antibody preparation.

A centrobin protein containing SEQ ID NO: 2 or 4, derivatives, fragments, analogs or homologs thereof, are utilized as immunogens in the generation of antibodies that immunospecifically-bind these protein components. The term “antibody” as used herein refers to immunoglobulin molecules and immunologically active portions of immunoglobulin molecules, i.e., molecules that contain an antigen binding site that specifically binds (immunoreacts with) an antigen, such as centrobin. Such antibodies include, polyclonal, monoclonal, chimeric, single chain, F_{ab} and F(_{ab}′)₂ fragments, and an F_{ab} expression library. Various procedures known within the art may be used for the production of polyclonal or monoclonal antibodies to a centrobin protein sequence of SEQ ID NO: 2 or 4, or a derivative, fragment, analog or homolog thereof.

For the production of polyclonal antibodies, various suitable host animals (e.g., rabbit, goat, mouse or other mammal) are immunized by injection with the native protein, or a synthetic variant thereof, or a derivative of the foregoing. An appropriate immunogenic preparation contains, for example, recombinantly expressed centrobin protein or a chemically synthesized centrobin polypeptide. The preparation further includes an adjuvant. Various adjuvants used to increase the immunological response include, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants such as Bacille Calmette-Guerin and Corynebacterium parvum, or similar immunostimulatory agents. If desired, the antibody molecules directed against centrobin are isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of centrobin. A monoclonal antibody composition thus typically displays a single binding affinity for a particular centrobin protein with which it immunoreacts. For preparation of monoclonal antibodies directed towards a particular centrobin protein, or derivatives, fragments, analogs or homologs thereof, any technique that provides for the production of antibody molecules by continuous cell line culture may be utilized. Such techniques include, but are not limited to, the hybridoma technique (see Kohler & Milstein, 1975 Nature 256: 495-497); the trioma technique; the B-cell hybridoma technique (see Kozbor, et al., 1983 Immunol Today 4: 72) and the EBV hybridoma technique to produce monoclonal antibodies (see Cole, et al., 1985 In: MONOCLONAL ANTIBODIES AND CANCER THERAPY, Alan R. Liss, Inc., pp. 77-96). Monoclonal antibodies may be utilized in the practice of the present invention and may be produced by using hybridomas (see Cote, et al., 1983. Proc Natl Acad Sci USA 80: 2026-2030) or by transforming B-cells with Epstein Barr Virus in vitro (see Cole, et al., 1985 In: MONOCLONAL ANTIBODIES AND CANCER THERAPY, Alan R. Liss, Inc., pp. 77-96). Each of the above citations are incorporated herein by reference in their entirety.

Production of centrobin-specific single-chain antibodies are made according to known methods. (see e.g., U.S. Pat. No. 4,946,778). In addition, methodologies can be adapted for the construction of F_{ab} expression libraries (see e.g., Huse, et al., 1989 Science 246: 1275-1281) to allow rapid and effective identification of monoclonal F_{ab} fragments with the desired specificity for a centrobin protein or derivatives, fragments, analogs or homologs thereof. Non-antibodies can be “humanized” by techniques well known in the art. See e.g., U.S. Pat. No. 5,225,539. Antibody fragments that bind to a centrobin protein may be produced by techniques known in the art including, but not limited to: (i) an F(_{ab}′)₂ fragment produced by pepsin digestion of an antibody molecule; (ii) an F_{ab}, fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-centrobin antibodies, such as chimeric and humanized monoclonal antibodies, comprising both human and non-human portions, are made using standard recombinant DNA techniques. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better et al. (1988) Science 240:1041-1043; Liu et al. (1987) PNAS 84:3439-3443; Liu et al. (1987) J Immunol. 139:3521-3526; Sun et al. (1987) PNAS 84:214-218; Nishimura et al. (1987) Cancer Res 47:999-1005; Wood et al. (1985) Nature 314:446-449; Shaw et al. (1988) J Natl Cancer Inst 80:1553-1559); Morrison (1985) Science 229:1202-1207; Oi et al. (1986) BioTechniques 4:214; Jones et al. (1986) Nature 321:552-525; Verhoeyan et al. (1988) Science 239:1534; and Beidler et al. (1988) J Immunol 141:4053-4060. Each of the above citations are incorporated herein by reference in their entirety.

### Centrobin Recombinant Expression Vectors and Host Cells

Another aspect of the invention pertains to vectors, preferably expression vectors, containing a nucleic acid encoding centrobin protein. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. “Plasmid” and “vector” are used interchangeably as the plasmid is the most commonly used form of vector. However, the invention is intended to include such other forms of expression vectors, such as viral vectors (e.g., replication-defective retroviruses, adenoviruses and adeno-associated viruses), which serve equivalent functions.

Recombinant expression vectors comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in, vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive or inducible expression of a nucleotide sequence in many types of host cell and those that direct expression of the nucleotide sequence only in certain host cells (e.g., tissue-specific regulatory sequences). The design of the expression vector depend on such factors as the choice of the host cell to be transformed, the level of expression of protein desired, etc. Expression vectors are introduced into host cells to produce proteins or peptides, including fusion proteins or peptides, encoded by nucleic acids as described herein.

Recombinant expression vectors direct expression of centrobin in prokaryotic or eukaryotic cells. For example, centrobin can be expressed in bacterial cells such as E. coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89). Another exemplary centrobin expression vector is a yeast expression vector. Examples of vectors for expression in yeast S. cer. include pYepSec1 (Baldari, et al., (1987) EMBO J 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.). Alternatively, centrobin is expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39). A nucleic acid may also be expressed in mammalian cells using a mammalian expression vector. Examples of mammalian expression vectors include pCDM8 (Seed (1987) Nature 329:840) and pMT2PC (Kauffman et al. (1987) EMBO J. 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells, see, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

A recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev 3:537-546).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd Ed., Cold Spring Harbor Laboratory, Cold Spring Harbor Laboratory Press, Cold Spring Harbor, N.Y., 1989), and other laboratory manuals.

### Methods of Inhibiting Cell Growth

The growth of cells are inhibited by contacting a cell with or administering to an animal a composition containing a centrobin siRNA. The cell is further contacted with a transfection agent. Suitable transfection agents are known in the art. By inhibition of cell growth is meant the cell proliferates at a lower rate or has decreased viability compared to a cell not exposed to the composition. Cell growth is measured by methods know in the art such as, the MTT cell proliferation assay.

The term “small interfering RNA” (“siRNA”), also referred to in the art as “short interfering RNAs,” refers to an RNA (or RNA analog) comprising between about 10-60 nucleotides (or nucleotide analogs) that is capable of directing or mediating RNA interference. The term “siRNA” includes both double stranded siRNA and single stranded siRNA. Generally, as used herein the term “siRNA” refers to double stranded siRNA (as compared to single stranded or antisense RNA). The term “short hairpin RNA” (“shRNA”) refers to an siRNA (or siRNA analog) precursor that is folded into a hairpin structure and contains a single stranded portion of at least one nucleotide (a “loop”), e.g., an RNA molecule that contains at least two complementary portions hybridized or capable of hybridizing to form a double-stranded (duplex) structure sufficiently long to mediate RNAi (as described for siRNA duplexes), and at least one single-stranded portion, typically between approximately 1 and 10 nucleotides in length that forms a loop connecting the regions of the shRNA that form the duplex portion. The duplex portion may, but typically does not, contain one or more mismatches and/or one or more bulges consisting of one or more unpaired nucleotides in either or both strands. Without wishing to be bound by theory, shRNAs are processed into siRNAs by the conserved cellular RNAi machinery. shRNAs are capable of inhibiting expression of a target transcript that is complementary to a portion of the shRNA (referred to as the antisense or guide strand of the shRNA). In general, the features of the duplex formed between the guide strand of the shRNA and a target transcript are similar to those of the duplex formed between the guide strand of an siRNA and a target transcript. In certain embodiments of the invention the 5′ end of an shRNA has a phosphate group while in other embodiments it does not. In certain embodiments of the invention the 3′ end of an shRNA has a hydroxyl group.

Binding of the siRNA to an centrobin transcript in the target cell results in a reduction in centrobin production by the cell. The length of the oligonucleotide is at least 10 nucleotides and may be as long as the naturally-occurring centrobin transcript. Preferably, the oligonucleotide is 19-25 nucleotides in length. Most preferably, the oligonucleotide is less than 75, 50, or 25 nucleotides in length. For example, the centrobin siRNA includes the nucleotides at positions 1187-1209 (aaggatggttctaagcatatctt), 753-775 (aacccgcctggaacaaagccttt), 1199-1221 (aagcatatctttgagatggaaag), 1202-1224 (catatctttgagatggaaagtgt), 1285-1307 (cattcctggcgctggctcagaga), 1302-1324 (cagagagacgggaagaggactcc), 1304-1326 (gagagacgggaagaggactcctt), 1308-1330 (gacgggaagaggactcctttgac), 1313-1335 (gaagaggactcctttgacagtga), or 1314-1336 (aagaggactcctttgacagtgat) of SEQ ID NO:1. Centrobin siRNAs that hybridize to target mRNA decrease or inhibit production of the centrobin polypeptide product encoded by the centrobin gene by associating with the normally single-stranded mRNA transcript, thereby interfering with translation and thus, expression of the protein. Exemplary nucleic acid sequence for the production of centrobin siRNA include the sequence of SEQ ID NO: 5.

The centrobin siRNA is directed to a single target centrobin gene sequence. Alternatively, the siRNA is directed to multiple target centrobin gene sequences. For example, the composition contains centrobin siRNA directed to two, three, four, or five or more centrobin target sequences. By centrobin target sequence is meant a nucleotide sequence that is identical to a portion of the centrobin gene or complementary to a portion of a naturally occurring centrobin gene. The target sequence can include the 5′ untranslated (UT) region, the open reading frame (ORF) or the 3′ untranslated region of the human centrobin gene. Alternatively, the siRNA is a nucleic acid sequence complementary to an upstream or downstream modulator of centrobin gene expression. Examples of upstream and downstream modulators include a transcription factor that binds the centrobin gene promoter.

The cell is any cell that expresses centrobin. The cell is a breast cell, a pancreatic cell, an ovarian cell, a prostate cell, a testicular cell, a stomach cell, or a skin cell. For example, the cell is a tumor cell such as a carcinoma, adenocarcinoma, blastoma, leukemia, myeloma, or sarcoma. The cell is a breast cancer cell, a pancreatic cancer cell, an ovarian cancer cell, a prostate cancer cell, a testicular cancer cell, a stomach cancer cell, or a skin cancer cell.

A centrobin siRNA is directly introduced into the cells in a form that is capable of binding to the mRNA transcripts. Alternatively, the DNA encoding the centrobin siRNA is in a vector. Vectors are produced for example by cloning a centrobin target sequence into an expression vector having operatively-linked regulatory sequences that flank the centrobin sequence in a manner that allows for expression (by transcription of the DNA molecule) of both strands. An RNA molecule that is antisense to centrobin mRNA is transcribed by a first promoter (e.g., a promoter sequence 3′ of the cloned DNA) and an RNA molecule that is the sense strand for the centrobin mRNA is transcribed by a second promoter (e.g., a promoter sequence 5′ of the cloned DNA). The sense and antisense strands hybridize in vivo to generate siRNA constructs for silencing of the centrobin gene. Alternatively, two constructs are utilized to create the sense and antisense strands of a siRNA construct. siRNAs are transcribed intracellularly by cloning the centrobin gene templates into a vector containing, e.g., a RNA pol III transcription unit from the smaller nuclear RNA (snRNA) U6 or the human RNase P RNA H1.

### Methods of Treating Tumors

Patients with tumors characterized as expressing an abnormal level of centrobin are treated by administering, e.g., centrobin siRNA. siRNA therapy is used to inhibit expression of centrobin in patients suffering from or at risk of developing, for example, breast cancer, pacreatic cancer, ovarian cancer, prostate cancer, testicular cancer, stomach cancer, or skin cancer. Such patients are identified by standard methods of the particular tumor type. Breast cancer and ovarian cancer are diagnosed for example, by tomography, ultrasound or biopsy.

Treatment is efficacious if the treatment leads to clinical benefit such as a reduction in expression of centrobin, or a decrease in size, prevalence, or metastatic potential of the tumor in the subject. When treatment is applied prophylactically, “efficacious” means that the treatment retards or prevents tumors from forming or prevents or alleviates a clinical symptom of the tumor. Efficaciousness is determined in association with any known method for diagnosing or treating the particular tumor type.

siRNA antisense or other nucleic acid-based therapy is carried out by administering to a patient an siRNA by standard vectors and/or gene delivery systems or peptide-based delivery systems. Suitable gene delivery systems include liposomes, receptor-mediated delivery systems, or viral vectors such as herpes viruses, retroviruses, adenoviruses and adeno-associated viruses, peptide-siRNA conjugates among others. A therapeutic nucleic acid composition is formulated in a pharmaceutically acceptable carrier. The therapeutic composition may also include a gene delivery system as described above. Pharmaceutically acceptable carriers are biologically compatible vehicles which are suitable for administration to an animal, e.g., physiological saline. A therapeutically effective amount of a compound is an amount which is capable of producing a medically desirable result such as reduced production of a centrobin gene product, reduction of cell growth, e.g., proliferation, or a reduction in tumor growth or tumor mass in a treated animal.

Parenteral administration, such as intravenous, subcutaneous, intramuscular, and intraperitoneal delivery routes, may be used to deliver centrobin siRNA compositions. The inhibitors are administered systemically or locally. For treatment of hepatic tumors, direct infusion into the portal vein is useful.

Dosages for any one patient depends upon many factors, including the patient's size, body surface area, age, the particular nucleic acid to be administered, sex, time and route of administration, general health, and other drugs being administered concurrently. Dosage for intravenous administration of nucleic acids is from approximately 10⁶ to 10²² copies of the nucleic acid molecule.

The polynucleotides are administered by standard methods, such as by injection into the interstitial space of tissues such as muscles or skin, introduction into the circulation or into body cavities or by inhalation or insufflation. Polynucleotides are injected or otherwise delivered to the animal with a pharmaceutically acceptable liquid carrier, e.g., a liquid carrier, which is aqueous or partly aqueous. The polynucleotides are associated with a liposome (e.g., a cationic or anionic liposome). The polynucleotide includes genetic information necessary for expression by a target cell, such as one or more promoters.

### Assays to Identify Therapeutic Composition

Based on the identification of centrobin and the affect of this polypeptide on cell proliferation, it is recognized that altered expression of centrobin causes or contributes to the onset and/or progression of a disease or disorder that is associated with altered centrobin expression, the invention further features assays, including animal-based, cell-based or cell free assays for identifying therapeutics, including centrobin inhibitory compounds. In one embodiment, a cell expressing centrobin is incubated in the presence of a test compound alone or in the presence of a test compound and another protein (such as BRCA2 or another centrobin-binding protein) and the interaction between centrobin and BRCA2 is detected, e.g., by using a microphysiometer (McConnell et al. (1992) Science 257:1906). A change in the interaction between centrobin and BRCA2 in the presence of the compound indicates that the compound is useful to inhibit a disease or disorder that is associated with altered centrobin expression, such as cancer.

Cellular or cell-free assays can also be used to identify compounds which modulate expression of centrobin or centrobin activity, or which modulate the stability of a centrobin mRNA or protein. Accordingly, in one embodiment, a cell which is capable of producing centrobin is incubated with a test compound and the amount of protein produced in the cell medium is measured and compared to that produced from a cell which has not been contacted with the test compound.

### Centrobin: A Daughter Centriole-Associate Protein Required for Centriole Duplicates

Centrobin contains a large coiled-coil domain and is a core component of the centrosomes. Through detailed analysis of centrobin localization during different phases of the cell cycle in 76NTert cells, NIH3T3 cells with a primary cilium, U2OS cells treated with HU, immunogold Electron microscopy, it has been established that centrobin asymmetrically localizes to the daughter centrioles. Previously, only PARP-3 has been reported to localize preferentially to the daughter centriole. Centrobin binds to BRCA2 polypeptides and other binding partners of centrobin are proteins that preferentially localize to the daughter centrioles.

Depletion of centrobin in HeLa cells resulted in a high percentage of both interphase cells and mitotic cells having two, one, or no centrioles, even under the condition of S-phase arrest, demonstrating that centrobin depletion inhibits the duplication of centrioles. Further analysis in U2OS cells arrested by HU treatment confirms that inhibition of centriole duplication induced by centrobin depletion is not a consequence of cell-cycle arrest. Thus, centrobin is required for centriole duplication. CDK2 activity has been shown to be required for centriole duplication, and centrobin is a downstream target of CDK2. Thus, centrobin inhibitory compounds are useful in diseases characterized by alterations in CDK2 signaling pathways.

The present invention also demonstrates that γ-tubulin localization to the centrosomes is not affected in centrobin depleted cells. Furthermore, centrobin depletion does not affect microtubule organization and nucleation in interphase cells. Centrobin also plays a role in the assembly of mitotic spindles.

Inhibition of centriole duplication by centrobin depletion leads to lengthening of mitosis and failure of cytokinesis in a substantial population of cells. The impairment of cytokinesis induced by centrobin depletion is similar to the phenotypes observed in cells when centrosomes were micro-surgically removed or laser-ablated. In those studies, the majority of acentrosomal cells were still able to undergo mitosis but took significantly longer to complete cytokinesis with a high percentage of cells failing to complete cytokinesis. Centrobin directly effects cytokinesis, and lack of centrobin results in impaired cytokinesis due to formation of acentrosomal spindles.

Thus, centrobin, among a few known centrosome proteins, has a particularly important function in orchestrating centriole duplication.

The following materials and methods were used to characterize the centrobin compositions described herein.

Cells and media. MCF-7, T47D, U2OS, NIH3T3, and HeLa cells were grown in α-MEM (Life Technologies, Grand Island, N.Y.) supplemented with 10% fetal calf serum (FCS, Hyclone, Logan, Utah). COS-7 and 293T cells were grown in DMEM (Life Technologies, Grand Island, N.Y.) supplemented with 10% fetal calf serum. Breast epithelial cell strain 76N cells, its immortal derivative 76NTert cells, and MCF10A cells were grown in DFCI-1 medium.

Yeast two-hybrid constructs and screening. The cDNA encoding the C-terminal 1026 residues of BRCA2 was cloned into pGBKT7 (Clontech, Calif.) to generate the bait plasmid, pGBKT7-BRCA2-C3. Yeast two-hybrid screening was performed according to standard methods.

Plasmid constructs and SiRNA. The centrobin fragment isolated from the yeast two hybrid library was cloned into a modified pSG5 vector, pGEX2TK and pPROEX Hta to generate Myc-centrobin-C, GST-centrobin-C, and His-centrobin-C. The full-length-untagged, Myc-tagged, and GFP-tagged centrobin were constructed by PCR and restriction splicing. The full-length centrobin constructs (untagged, myc-tagged, and GFP-tagged) used in this study represent centrobin-α. The centrobin-C construct used in this study contains the extra 66 bp of centrobin β. It is referred to only as centrobin elsewhere in this paper.

siRNAs were synthesized by Dharmacon, Inc. The sequence of centrobin siRNA #1 was AGUGCCAGACUGCAGCAACTT (SEQ ID NO: 5), the centrobin siRNA #2 was CAACUGGACAAGACCCUGGTT (SEQ ID NO: 7). The sequence of FITC-GFP siRNA was GGCTACGTCCAGGAGCGCACC (SEQ ID NO: 8) and the scrambled siRNA was CAGTCGCGTTTGCGACTGG (SEQ ID NO: 9). The siRNA transfection was performed with Oligofectamine (Invitrogen, Inc.) according to the manufacturer's instructions.

Immunofluorescence. Cells grown on coverslips (Fisher) were fixed in 3.7% paraformaldehyde/PBS for 10 min at room temperature, permeabilized in 0.5% Triton X-100/PBS, blocked with 10% goat serum in PBS, and incubated with anti-centrobin serum, anti-Myc (9E10), anti-α-tubulin or anti-γ-tubulin (Sigma) in 10% goat serum/PBS. The antibody complexes were detected with FITC or rhodamine-conjugated goat anti-rabbit IgG or goat anti-mouse IgG (Jackson Laboratory) and the DNA was stained with DAPI. Cells were also fixed in ice-cold methanol for 10 min, in which case the permeabilization step was eliminated. A fixation solution of 0.5% glutaraldehyde was also used. An extraction step before fixation with 0.5% Triton X-100 in 80 mM Pipes, 1 mM MgCl₂, 5 mM EGTA, pH 6.8 was added in some experiments as indicated. Gray level images were acquired using an ORCA-ER CCD camera mounted on a Nikon epifluorescence microscope, and pseudocolored using Adobe Photoshop software.

Electron Microscopy. For immuno-EM, centrosome and nuclear complexes were enriched according to the known methods. Briefly, 70%-80% confluent HeLa cells were scraped off the culture dishes and pelleted. The cells were resuspended in 10 volumes of distilled water for 1 min, then added an equal volume of lysis buffer (2 mM Hepes 4% Triton X-100) and incubated for 5 more min. Equal volume of 8% paraformaldehyde plus 1% glutaraldehyde was then added to fix the sample for 15 min at room temperature. The fixed samples were washed for three times with Hepes buffer (Hepes 2 mM, pH 7.4), then dehydrated and embedded in LR white resin (Ted Pella, Inc.). Thin sections were blocked in blocking buffer (2 mM Hepes, 1% fish gelatin, 0.4% Triton X-100) at room temperature for 1 h, then incubated with anti-centrobin antibody in blocking buffer (0.5 μg/ml) at 4° C. overnight, followed by incubation with goat anti-rabbit IgG conjugated with 10 nm-gold particles (Ted Pella, Inc.) for 2 h at room temperature. The thin sections were further stained with saturated aqueous uranyl acetate and analyzed using a Philips CM-10 transmission electron microscope. To obtain serial thick sections, the purified nuclear-centrosome complexes were fixed in 2.5% glutaraldehyde for 30 min, followed by incubation in 2% osmium tetroxide for 10 min, then in 2% uranyl acetate for another 10 min, embedded in Embed 812. The samples were serially thick-sectioned at 250 nm or 500 nm, analyzed using a JEOL-1234 transmission electron microscopy. A total of 58 centrobin knockdown cells were examined, of which 47 cells were examined using 500 nm sections and 11 cells were examined using 250 nm sections. A total of 33 control cells were examined using 500 nm sections.

Time-lapse imaging. HeLa cells plated on 35-mm dishes glass with coverslips bottom were transfected with control siRNA or centrobin siRNA. Eight hours after transfection, the medium was replaced with. CO2-independent medium (Invitrogen, CA). The cells were imaged every 5 min for 48 h using a 20× phase contrast lens on a Nikon TE2000-U inverted microscope equipped with an ORCA-ER CCD camera (Hamamatsu) controlled by Metamorph software (Universal Imaging Corp.). The microscope is enclosed in an incubator box (Life Imaging Services, Switzerland) to maintain the temperature at 37° C.

### Example 1

### Identification, Expression, and Characterization of Centrobin

Centrobin was identified in a yeast two-hybrid screening with the conserved C-terminal 1026 residues of BRCA2 as bait. One set of 11 positive clones encoded the C-terminal 539 amino acids and the 3′-untranslated region of a novel protein designated centrobin (centrosomal BRCA2 interacting protein) (FIGS. 1C and 1E). Northern blotting revealed a single centrobin mRNA transcript of approximately 3.8 kb expressed in most human tissues and all of the cell lines tested, although the levels varied (FIGS. 1A, B).

Two rounds of 5′ RACE (rapid amplification of cDNA ends) PCR, using the human mammary gland Marathon-ready cDNA (Clontech, Calif.) as a template, allowed the cloning of the 5′ end of centrobin cDNA and to assemble a 3,718-bp cDNA predicting a 903-aa polypeptide (FIG. 1C). The size of this cDNA, as well as the presence of several in-frame stop codons 5′ to the first in-frame methionine, indicate that this cDNA represents the 3.8-kb transcript observed on the Northern blotting (FIGS. 1A, B). One cDNA library-derived clone revealed a 66 bp in-frame insertion after nucleotide 3433, predicating a 925-aa polypeptide (FIG. 1D). This clone appears to represent a minor transcript, given that only one of the nine sequenced clones contained this 66 bp-insertion. In addition, only one EST clone in the GenBank contains this insertion (GenBank Acc: BM127046). The 3718 bp transcript was designated as centrobin α (GenBank Acc: AY160226) and the 3,784 bp transcript as centrobin α (GenBank Acc: AY160227). A search of the NCBI database indicated that centrobin is a unique protein. Weak homology was observed between the central region of centrobin and various coiled-coil proteins. Centrobin is predicted to have a coiled-coil region in its middle and non-coiled regions at the C- and N-termini, as do some known centrosomal proteins such as pericentrin and ninein (FIG. 1E).

The expression and function of centrobin, was examined by generating an anti-centrobin antiserum against a His-tagged fusion protein of the C-terminal 539 aa of centrobin. To determine whether this antiserum specifically recognizes recombinant and endogenous centrobin, cell lysates from various cell lines and 293T cells transfected with vector, Myc-tagged centrobin, or GFP-tagged centrobin were analyzed by Western blotting using affinity purified anti-centrobin antibody. Consistent with the size of centrobin, anti-centrobin antibody recognized a 100-kDa protein in all cell lines tested (FIG. 1F, lanes 3-9). It was found that the level of this 100 kDa protein is significantly higher in the Myc-centrobin-transfected 293T cells than in the vector-transfected cells (FIG. 1F, compare lanes 3 and 4). Anti-Myc antibody specifically detected the 100-kDa band only in the myc-centrobin-transfected cells (lane 2). The specificity of the antiserum was also demonstrated by the presence of a 127-kDa protein in the GFP-centrobin-transfected 293T cells (lane 5). Furthermore, preincubating the antibody with purified His-centrobin significantly diminished the specific signal detected by this antiserum. These results demonstrate that the anti-centrobin antiserum specifically detects endogenous centrobin and that centrobin is ubiquitously expressed (FIG. 1F).

### Example 2

### Localization of Centrobin to the Centrosome

The anti-centrobin antibody characterized above (FIG. 1F) was used to examine the localization of endogenous centrobin in a normal human mammary epithelial cell line (76N) and several cancer cell lines (T47D, MCF-7 and Capan-1). A typical centrosomal staining pattern was observed in all the cell lines tested, with one or two perinuclear dots in the interphase cells (FIG. 2A g, o, s) and a single focus at the end of each mitotic spindle in mitotic cells (FIG. 2A c, k). The centrobin staining pattern was similar to that of γ-tubulin, a protein known to specifically localize to centrosomes (FIG. 2A d, h, p, t). An identical centrosomal staining pattern was observed in MCF10A, HeLa, COS-7, and 293T cells. The centrosomal staining was observed under three different fixation conditions (3.7% formaldehyde, 100% methanol, or 0.5% glutaraldehyde) and also when cells were extracted with 0.5% Triton X-100 in 80 mM PIPES, 1 mM MgCl2, and 5 mM EGTA, pH 6.8 before fixation. Furthermore, the centrosomal localization of centrobin was not affected by treatment with Nocodazole. These findings strongly indicated that centrobin is likely to be a bona fide core component of the centrosomes. Importantly, GFP-centrobin and Myc-centrobin also localized to the centrosomes in the transfected cells when they were expressed at a very low level (FIGS. 2B, C). It is notable that both GFP-centrobin and Myc-centrobin formed bundle-like structures when expressed at high levels, which is likely to be an artifact of high-level expression since the endogenous centrobin is expressed at very low levels and is found mainly on centrosomes. A truncated Myc-tagged centrobin (centrobin-C, encoding the C-terminal 539 aa) also localized to the centrosomes when expressed at low levels (FIG. 2D), indicating that the C-terminal 539 aa of centrobin is sufficient for centrosomal localization. It is noteworthy, that when Myc-centrobin-C was expressed at a high level, it decorated the microtubules, which is also likely to be an artifact of high-level expression.

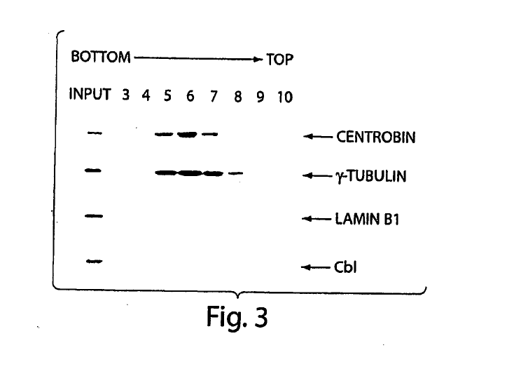

To corroborate the observation using immunofluorescence analysis that centrobin localizes to the centrosomes, the centrosomes from 293T cells were biochemically purified using sucrose-gradient sedimentation and analyzed its composition by Western blotting. As shown in FIG. 3, centrobin was found to be in the fractions expected to contain the centrosomes as confirmed by the presence of γ-tubulin in these fractions (FIG. 3, upper and second panel). Similar data were obtained using MCF7 cells, the human breast cancer cells. The centrosomal fractions were shown to be free of nuclear and cytoplasma contamination by Western blotting using Lorain B1 as a nuclear marker and Cb1 as a cytoplasmic marker (FIG. 3, lower two panels). Collectively, the immunofluorescence and biochemical studies unequivocally demonstrated that centrobin is a centrosomal protein.

### Example 3

### Centrobin Localizes to Daughter Centrioles

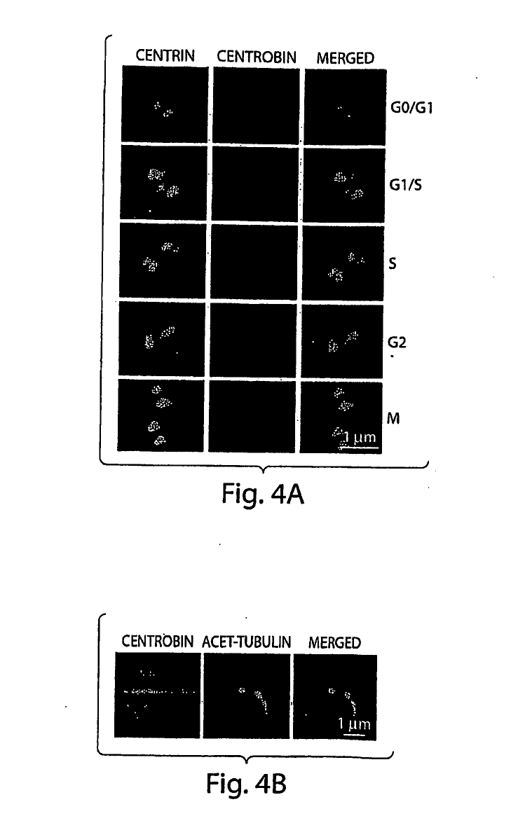

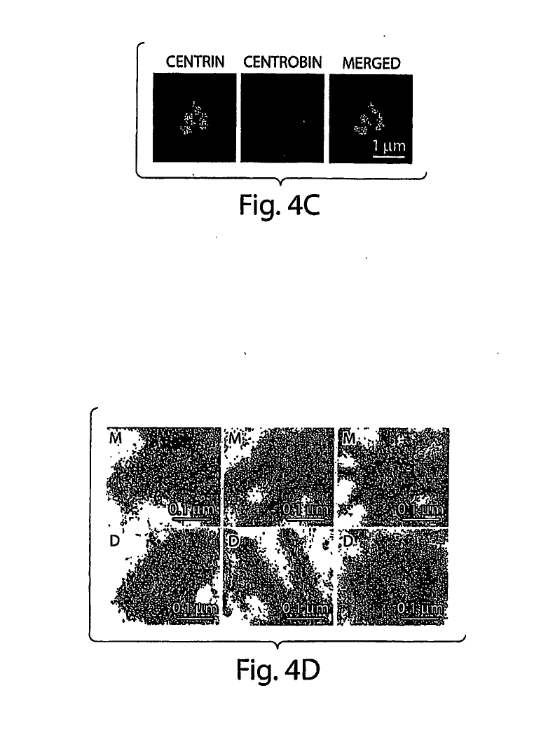

During immunolocalization analyses it was noted that the staining pattern of centrobin differed in cells that appeared to be at different phases of the cell cycle, suggesting the possibility that centrobin may differentially localize in either the mother or daughter centrioles. Centrobin localization experiments were performed in synchronized 76NTert cells (a hTert immortalized cell line derived from normal human mammary epithelial 76N cells), in which the two centrioles typically are located farther from each other than they are in other cells. Synchronization was achieved by mitotic shake-off. A majority of cells at G0/G1 exhibited a strongly stained centriolar dot with the other centriole stained weakly or not at all with anti-centrobin antibody. Superposing centrin and centrobin staining demonstrated essentially complete correspondence of weak centrin staining (daughter centriole) with strong centrobin staining and vice versa (FIG. 4A). In the majority of G1/S, S and G2/M phase cells, there are usually two strongly stained centrobin dots, correlating with the two newly synthesized daughter centrioles, as indicated by the weaker centrin-2 staining. In some cells, three or four centrobin dots can also be found, including one or two dots with weaker centrobin staining, which is likely to correlate with the original daughter centrioles (mother centrioles, in the current duplication cycle). These findings indicate that centrobin is preferentially incorporated into the newly assembled daughter centriole during centriole assembling at the late G1 or early S phase and that centrobin remains in the daughter centrioles throughout the cell cycle. At the next cycle of centriole duplication, the amount of centrobin on the original daughter centriole eventually decreases as shown in the G1/S and S phase cells (FIG. 4A).

To corroborate these observations, double-immunofluorescence localization of centrobin and acetylated-α-tubulin was performed in NIH-3T3 cells. Previous studies have shown that in G0/G1 phase a primary cilium grows from the mother centriole in NIH-3T3 cells and that this cilium can be stained with anti-acetylated-α-tubulin antibody. As shown in FIG. 4B, anti-centrobin staining clearly superimposed with the centriole without the primary cilium, the daughter centriole. U2OS cells treated with Hydroxy Urea (HU) for 72 h were also analyzed, a treatment known to induce extensive centrosome amplification. As shown in FIG. 4C, anti-centrobin staining always superimposed with the centrioles with the weaker centrin staining, the newly synthesized centrioles (FIG. 4C).

To further characterize the localization of centrobin on the centrioles, immunogold electron microscopy was performed. Nuclear-centrosome complexes were prepared and embedded in LR white resin. Thin sections were incubated with anti-centrobin antibody, followed by incubation with gold-conjugated anti-rabbit secondary antibodies. As in the immunofluorescence studies, centrobin clearly localized to the daughter centrioles, the centrioles without the characteristic appendage structure of mother centriole (FIG. 4D). It was found that centrobin mainly localizes outside the triplet microtubule blades of the daughter centriole (FIG. 4D). Some centrobin staining was also found in the lumen and on the triplet microtubule blades of daughter centriole (FIG. 4D). Taken together, these results strongly indicate that centrobin localizes preferentially to the daughter centriole.

### Example 4

### Centrobin is Required for Centriole Duplication

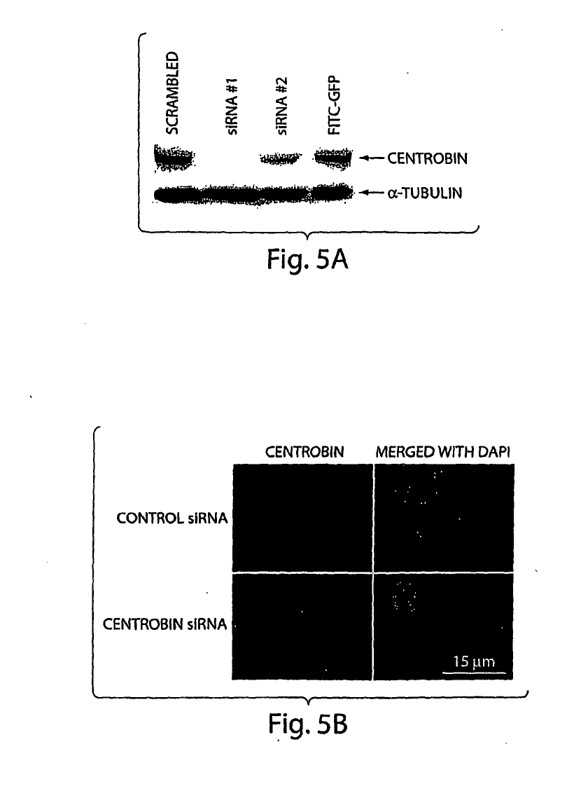

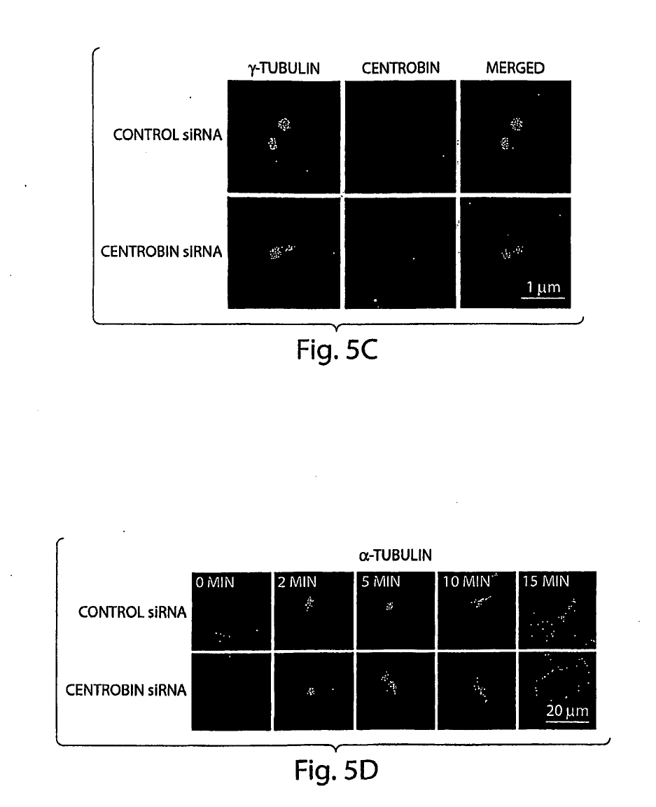

siRNAs targeting the coding region of centrobin were used to knock down centrobin expression. As shown in FIG. 5A, the endogenous centrobin level was markedly reduced after centrobin siRNA transfection of HeLa cells, but not after the scrambled siRNA and GFP siRNA were transfected (FIG. 5A, upper panel). Transfection of HeLa cells with FITC-labeled GFP-siRNA indicated a transfection efficiency of about 90%. Densitometry analysis of the Western blots indicated that 80% of centrobin was reproducibly depleted with centrobin RNAi#1, which were used in all subsequent experiments. The reduction of centrobin levels was also confirmed by immunofluorescence analysis (FIG. 5B). Immunofluorescence analysis with anti-γ-tubulin antibodies revealed that the γ-tubulin staining pattern was not visibly altered in the HeLa cells with undetectable levels of centrobin (FIG. 5C). Furthermore, no gross abnormalities in microtubule nucleation and organization was observed in centrobin-depleted cells (FIG. 5D), suggesting that centrobin likely does not substantially contribute to microtubule organization and nucleation, at least in interphase cells.

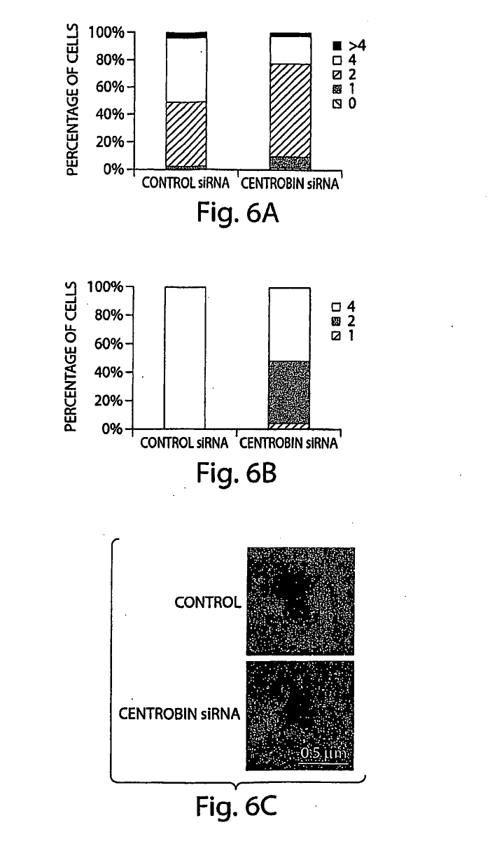

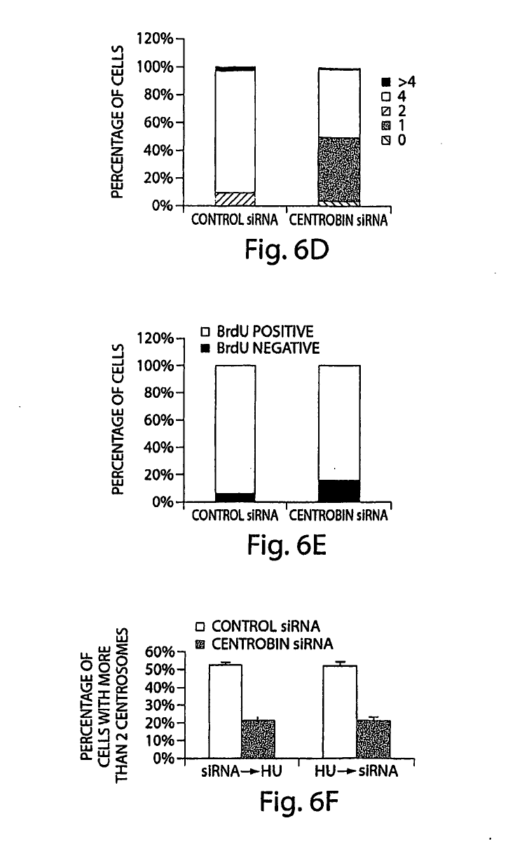

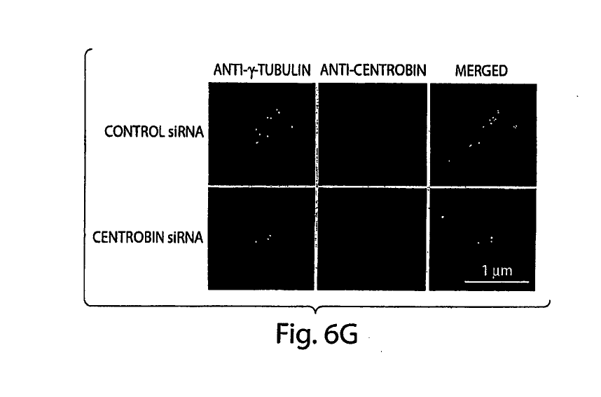

To examine the function of centrobin in centriole duplication, differences in the numbers the centrioles in control RNAi-treated versus centrobin-depleted HeLa cells were examined using anti-centrin-2 staining. In centrobin-depleted cultures, 21% of interphase cells had four centrioles, 67% had two centrioles, 7% had one centriole, and 2% had no centriole (FIG. 6A). In contrast, among the control HeLa cells, 47% of the interphase cells had four centrioles and 47% had two centrioles, only 2% of interphase cells had one centriole, and 0.3% had no centriole (FIG. 6A). Thus, centrobin depletion induced a marked reduction in the proportion of cells with four centrioles and, correspondingly, an increase in the proportion of cells with fewer than four centrioles. An even more pronounced difference was seen in mitotic cells. Essentially all mitotic cells in the control culture had four centrioles; in contrast, only 51% of the centrobin-depleted mitotic cells had four centrioles, whereas 45% had two and 4% had one centriole (FIG. 6B). Thus, RNAi-mediated centrobin depletion dramatically inhibited centriole duplication. Nevertheless, the cells were still able to progress through the cell cycle, at least once or twice, generating cells with one centriole or none.

To confirm that the anti-centrin-2 staining dots indeed represent centrioles, the cells were co-stained with anti-α-tubulin and anti-centrin-2 after the cells were cold-treated and detergent-extracted to depolymerize the microtubules. It was found that anti-centrin-2 staining correlated well with anti-α-tubulin staining. And the centriole number assessed by anti-α-tubulin staining correlated with the centriole number assessed by anti-centrin-2 staining. The number of centriole in control and centrobin-depleted cells was also examined using electron microscopy. By examining consecutive thick sections spanning whole nucleus-centrosome complexes, out of 58 centrobin depleted cells examined, 1 cell had five centrioles, 15 cells had four centrioles, 34 cells had two centrioles, 5 cells had one centriole, and 3 cells had no centriole. A total of 33 control cells were also examined, with 1 cell had six centrioles, 19 cells had four centrioles, 13 cells had two centrioles, and no cells had one centriole or zero centriole. This finding is similar to what was observed using anti-centrin-2 staining. Cells with one or no centriole were observed in centrobin-depleted cells but not in control cells, which indicates that centrobin depletion inhibited centriole duplication, confirming the finding using anti-centrin-2 staining dots to represent centrioles.

Centrobin depletion directly inhibits centriole duplication, as demonstrated by the effect of centrobin depletion on HeLa cells arrested in the S phase. Forty-eight hours after siRNA transfection, the cells were exposed to HU for 24 h to induce the S phase arrest. The cells were incubated for 30 min with BrdU, harvested, and stained with anti-BrdU and anti-centrobin antibodies. BrdU staining demonstrated that 94% of the control cells and 85% of the centrobin-depleted cells were arrested in the S phase, indicating that centrobin-depleted cells are still able to enter S phase (FIG. 6D). Since centriole duplication occurs during the late G1 and S phases, all cells arrested in the S phase should contain four centrioles. Indeed, a majority (about 87%) of the control RNAi-transfected cells contained four centrioles (FIG. 6E); only 9% of these cells had fewer than four centrioles. In contrast, only 48% of the centrobin-depleted cells contained four centrioles, while 50% contained fewer than four centrioles (vs 9% of control cells) (FIG. 6E). These experiments demonstrated that the majority of centrobin-depleted cells are able to enter the S phase, but are unable to undergo centriole duplication. Therefore, the inhibition of centriole duplication upon centrobin depletion is not a consequence of cell-cycle arrest.

Furthermore, the effect of centrobin depletion in U2OS cells was examined. It has been reported that, upon prolonged S-phase arrest by HU, the centrosomes in these cells become overamplified. If the effect of centrobin depletion on centrosome duplication was a consequence of preventing cells from entering the S phase, then centrobin RNAi would be expected to be effective only when it was transfected into cells before HU treatment. On the other hand, if centrobin has a direct role in centrosome duplication, then centrobin RNAi would be expected to inhibit centriole duplication even when it is introduced into cells after S-phase arrest. As shown in FIG. 6F, centrosome overamplification was inhibited to a similar extent regardless of whether centrobin RNAi was introduced into U2OS cells before or after S-phase arrest. It was found that about 52% of the cells transfected with control siRNA had overamplified centrosomes, whereas only 21% of the cells transfected with centrobin siRNA had overamplified centrosomes. Taken together, these findings clearly indicated that centrobin is required for centriole duplication.

### Example 5

### Centrobin Depletion Leads to Impaired Cytokinesis

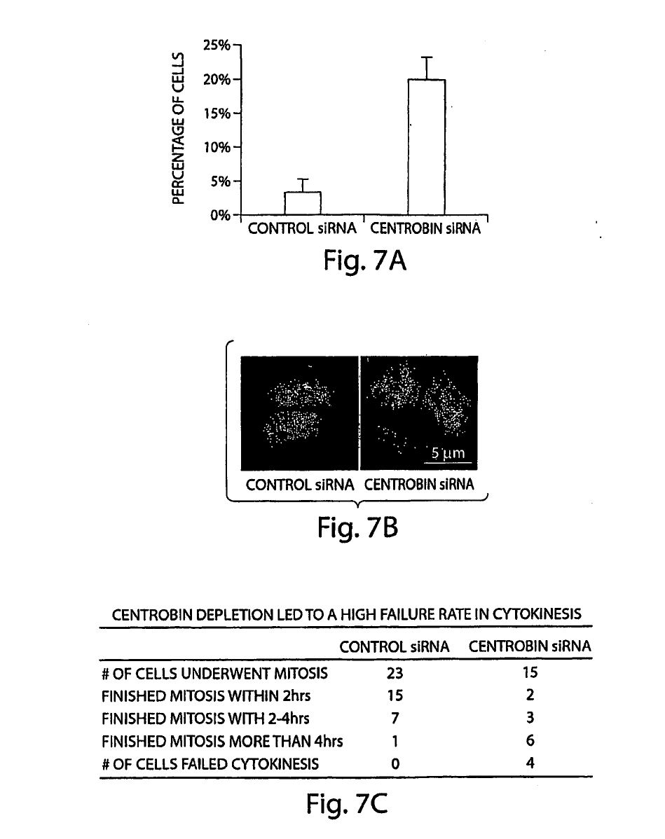

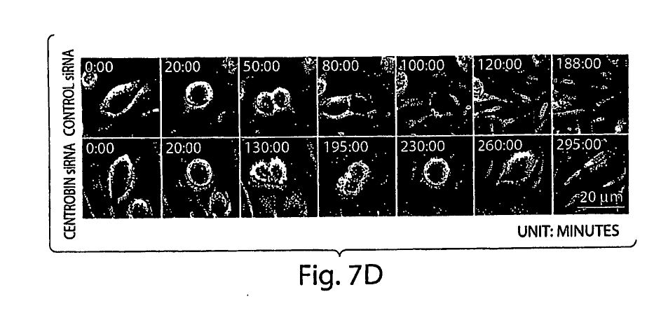

When centrobin was depleted in HeLa cells by the use of siRNA, it was observed that the percentage of cells with two or more nuclei increased significantly (from 3% in control siRNA-transfected cells to 20% in the centrobin-depleted cells), indicating a failure of cytokinesis in a proportion of the centrobin-depleted cells (FIGS. 7A, B). To further explore this finding, the progression of cell division was examined with time-lapse microscopy. For this purpose, HeLa cells were transfected with control or centrobin siRNA and phase contrast time-lapse microscopy was initiated 8 h later and continued for 48 h (see videos in supplementary data). In the control cultures, 15 of the 23 observed cells completed mitosis within 2 h, while 22 of 23 cells completed mitosis within 4 h; no cell failed to complete cytokinesis (FIGS. 7C, D). In contrast, out of the centrobin siRNA-transfected cells that went into mitosis, only 2 of 15 observed cells completed mitosis within 2 h and 5 of 15 cells completed mitosis within 4 h, while 6 of 15 cells failed to do so within 4 h and 4 of 15 cells exited mitosis without finishing cytokinesis. This finding indicated that centrobin depletion impaired cytokinesis.

### Example 6

### Depletion of Centrobin Induces Cell Death in Cancer Cells but not in Normal Cells

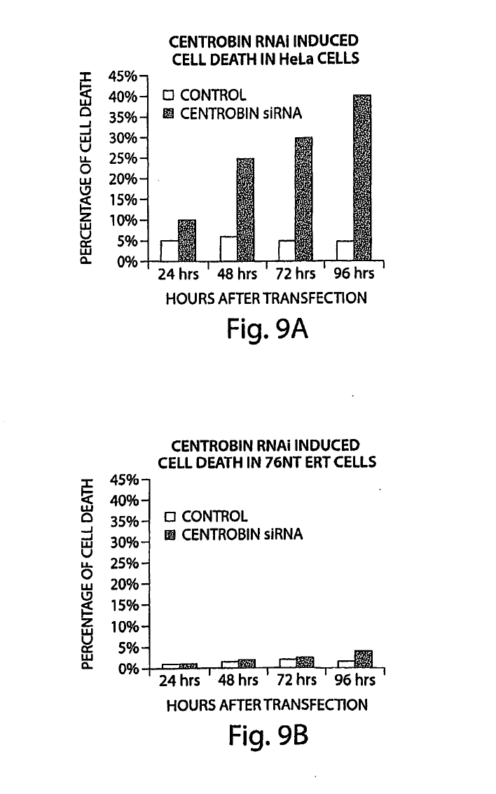

When centrobin is depleted using RNAi in cancer cells, including HeLa cells, U2OS cells and 293T cells, extensive cell death was observed by microscopic examination of the live cells or samples stained using DAPI. To quantify the extent of cell death induced by centrobin depletion, HeLa cells were transfected with centrobin RNAi or scrambled RNAi. The percent of cells that died was examined at different time points after transfection by typan blue staining. As shown in FIG. 9A, there are 20%, 25%, 30%, 40% cell death at 24 hrs, 48 hrs, 72 hrs and 96 hrs respectively when the cells were transfected by centrobin RNAi, while there are only about 5% cell death at all the time points when the cells were transfected by control RNAi.

In contrast, in normal cells such as 76N cells or 76Tert cells, negligible cell death was observed when centrobin was depleted. As shown in FIG. 9B, there is little difference in the amount of cell death in centrobin RNAi or scrambled RNAi transfected cells. About 1-1.5% or 1-4% cell death was observed in scrambled RNAi and centrobin RNAi transfected cells respectively.

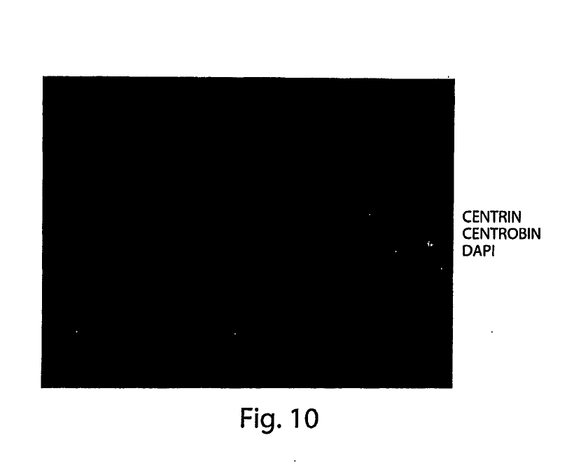

Depletion of centrobin inhibits centriole duplication in normal cells, leading to cells with split but unduplicated centrosomes. Centrobin depletion in HeLa cells inhibits the centriole duplication and led to cells with one, two, three or no centrioles, which is due to the uncoupling of centriole duplication and DNA replication. In non-transformed cells, since the centriole duplication is always coupled with cell cycle, the normal cells are arrested in G1 phase after centrobin knockdown. Indeed, 76Tert cells were arrested mainly in G1 phase after centrobin knockdown. Furthermore, the status of centriole was examined in these cells after centrobin depletion, and in the cells with centrobin depleted, there are usually two split unduplicated centrioles as shown in FIG. 10.

Inhibition of centrosome duplication by depleting centrobin induced cancer cell death while only inducing cell arrest in normal cells. These data indicate that centrobin inhibitors preferentially induce cell death of cancer cells compared to normal cells and is useful to reduce tumors.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated polypeptide at least 95% identical to the amino acid sequence of SEQ ID NO:2 or SEQ ID NO:4 or a fragment thereof at least 400 amino acids in length, wherein said polypeptide or fragment binds BRCA-2.

2. The polypeptide of claim 1, wherein said polypeptide is at least 99% identical to the amino acid sequence of SEQ ID NO:2 or SEQ ID NO:4.

3. The polypeptide of claim 1, wherein said polypeptide comprises a single conservative amino acid substitution relative to the amino acid sequence of SEQ ID NO:2 or SEQ ID NO:4.

4. The polypeptide of claim 1, wherein said fragment comprises amino acids 371-903 of SEQ ID NO:2.

5. An isolated nucleic acid molecule encoding the polypeptide of claim 1 or the complement of said nucleic acid molecule.

6. The nucleic acid molecule of claim 5, comprising the nucleic acid sequence of SEQ ID NO: 1 or SEQ ID NO: 3, or a nucleic acid at least 99% identical to the nucleic acid sequence of SEQ ID NO: 1 or SEQ ID NO: 3.

7. A nucleic acid vector comprising the nucleic acid molecule of claim 5.

8. An isolated host cell comprising the vector of claim 7.

9. An antibody that selectively binds to a centrobin polypeptide, and fragments, or derivatives of said antibody.

10. The antibody of claim 9, which selectively binds to a polypeptide comprising amino acids 371-903 of SEQ ID NO: 2.

11. A method of treating a subject suffering from or predisposed to a cancer, comprising administering to said subject a centrobin inhibitory compound.

12. The method of claim 11, wherein said cell is a cancer or tumor cell, or a stromal cell associated with a cancer or tumor cell.

13. The method of claim 11, wherein said centrobin inhibitory compound is selected from the group consisting of an siRNA, shRNA, antisense nucleic acid, and an antibody that selectively binds centrobin.

14. The method of claim 13, wherein the siRNA comprises the nucleic acid sequence of SEQ ID NO:5 or SEQ ID NO: 7.

15. The method of claim 13, wherein said centrobin inhibitory compound is administered in an amount sufficient to induce apoptotic cell death in one or more cells expressing centrobin.

16. The method of claim 12, wherein said cancer cell is a breast cancer cell, a pacreatic cancer cell, an ovarian cancer cell, a prostate cancer cell, a testicular cancer cell, a stomach cancer cell, or a skin cancer cell.

17. The method of claim 11, further comprising administering to said subject an anti-proliferative agent.

18. The method of claim 17, wherein said anti-proliferative agent is a chemotherapeutic compound.

19. A method for selecting an inhibitor of aberrant cell proliferation associated with a cell proliferative disease or disorder that is associated with an altered centrosome number comprising the steps of contacting a cell having altered centrosome number with a candidate compound; and selecting a compound that decreases centrobin expression, wherein a decrease in centrobin expression indicates that said compound inhibits aberrant cell proliferation.

20. The method of claim 19, wherein said candidate compound is an siRNA or an shRNA.


SEQUENCE LISTING (9 sequences)

SEQ ID NO: 1 - DNA, 3718 bp, Artificial Sequence
  Chemically Synthesized
gtggtctcgcgggcgggtgacgtcatcgaggcgagagaggcggagccgtggacagtgggcgggaggctgccaacggttttgagcgtagggggaggcgtgagagggggatctcaggggaggaggtcaatcgcttgccccccactttggcaaattggggactgaggactggaagggtggagagtaggcggaaccaggtggtcgtcggggcagaggatctcgggctaggcttgagggcggcgtgcttcttagggacgacttagggcgtgactgagggttcacaaggtttcttttggggtggtcgggagggagagattctagggaacaaggaagctcgctatggctttcttgccaggaggggtcgaagggaaagtacaagggagctgaccctgggtagaacgggtgaagggatgggggagcgtgaggttccgccctctcttgagactggaaccaattgagggactagtagggcagggggacagaaattgggctcctagtggatttgggtccgtttccgttgggacgttttgggtgtgagaacttaagagctcagttgaccggggatagcctgtgccggagttgatctgcagcttccagcactcgtagtcgggaagaggagcttcagcagcgctgttgtcccacagtaggtcttctgtccgcacccgctctgcgctgcaccctcttaacgctgttcccaggagctggggaaagggatgcttttgcccactcccatggcccctggaactggtggaaacctttcctctaaccagaaagcctcgatatccttaattcaccaaggatccttggcgtggagtcttcctcccttctcccaagtctttctccgtgaacttttcctcctggactttgctaaagcagaacctcccagctctttgctgtctccggttgtctcttccctgtattcatggcaacatcagctgacagccccagttcacccctcggggcggaggatctcctgagtgattcatcagaaccccctgggctcaaccaagtgtcgtctgaagtgacctcccagctctatgcttctttgcgcctcagccggcaggcggaggccacggcccgagcccagctgtatttaccctccacctccccgcctcatgaagggttagacggcttcgcccaagaattgagtcgaagcttgtcagtcggattggaaaagaacttgaagaaaaaggatggttctaagcatatctttgagatggaaagtgttcggggtcagctccagaccatgctccaaacctcacgtgatacagcctatcgggatcctctcattcctggcgctggctcagagagacgggaagaggactcctttgacagtgatagcacagccaccttgctcaacacccggcccctgcaagacttgtctccatctagctcagcccaagccctggaggagctgtttccccgctacaccagccttcggccagggcctccactcaatcccccagattttcaggggctgagagatgcattggattcagagcatacccgccgcaagcattgtgagcgccatattcagagcctgcagacccgagtgttagagctacagcaacaattagccgtggctgtggctgccgaccgcaagaaagataccatgattgaacaactggacaagaccctggcccgtgtggtggagggctggaaccggcatgaggctgagcggacagaggttctcaggggacttcaagaggaacaccaggcagcagagctcaccagaagcaagcagcaggagacagtaacccgcctggaacaaagcctttctgaggccatggaggccctgaatcgtgagcaggaaagtgccagactgcagcaacgggaaagagagacactggaggaggaaaggcaagctctgactctgaggttggaggcagaacagcagcggtgctgtgtcctgcaggaagagcgggatgcagctcgggctgggcaactgagtgagcatcgagagttggagactcttcgggctgccctagaagaagaacggcagacctgggcccagcaagagcaccagcttaaggaacactaccaggcgctgcaggaggagagccaggctcagctggaaagggagaaggagaagagccagagggaagcccaggccgcctgggagacccagcaccagttggcattggtgcagtctgaggtgcggcggctggaaggagagctggatacagctcggagagagagagatgccctgcagctggaaatgagcttggtgcaggcccggtatgaaagccagcggatccagctggagtcggagctggctgtgcagctggagcagcgggtgacagagcggctggcgcaggctcaggagagcagcctacggcaagcagcctccctcagggaacatcacaggaagcagctgcaggacctgagtggacagcaccagcaggagctggccagtcagctagctcagttcaaggtggaaatggcagaacgagaggaacggcaacagcaggtggctgaggactacgagctcagactggcccgggagcaagcgcgagtgtgcgaactgcagagtgggaaccagcagctggaggagcagcgggtggagctggtggaaagactgcaggccatgctgcaggcccactgggatgaggccaaccagctgctcagcaccactctcccgccgcccaaccctccagctcctcctgctggaccctccagccccgggcctcaggagcccgagaaggaggagaggagggtctggactatgcctcccatggccgtggccctgaagcctgtattgcagcagagccgggaagcaagggacgagctacctggagcgcctcctgttctttgcagttcctcctcagatcttagcctcctgttgggcccctcttttcagagccagcattctttccagcccctggagcccaaaccagacctcacttcatccacagctggggccttctctgcacttggggccttccatcccgatcatagggcagaaaggccattccctgaggaagatcctggacctgacggggagggcctcctaaagcaagggctgccgcctgctcagctggagggcctcaagaattttttgcaccagttgctggagacagtgccccagaacaatgagaacccttctgtcgacctgttgccccctaagtctggtcctctgactgtcccatcttgggaggaagcccctcaagtgccacgtattccaccgcctgtccacaaaaccaaagttcccttagccatggcatccagtcttttccgggtccctgagcctccctcctcccattcacaaggcagtggtcccagcagtggttccccagagagaggtggagatgggcttacattcccaaggcagctgatggaggtgtctcaactgttgcgactctaccaggctcggggctggggggctctgcctgctgaggatctcctgctctacctgaagaggctggaacacagcgggactgatggccgaggggataatgtccccagaaggaacacagactcccgcttgggtgagatcccccggaaagagattccctcccaggctgtccctcgccgccttgctacagcccccaagactgaaaaacctcccgcacggaagaaaagtgggcaccctgccccgagtagcatgaggagccgggggggagtctggagatgagcccccctaccctctctcctctttgttctctcattgttgttattttaataaatgctcattagtctgcaaaaaaaaaaaaaaaaaaaa

SEQ ID NO: 2 - protein, 903 aa, Artificial Sequence
  Chemically Synthesized
MATSADSPSSPLGAEDLLSDSSEPPGLNQVSSEVTSQLYASLRLSRQAEATARAQLYLPSTSPPHEGLDGFAQELSRSLSVGLEKNLKKKDGSKHIFEMESVRGQLQTMLQTSRDTAYRDPLIPGAGSERREEDSFDSDSTATLLNTRPLQDLSPSSSAQALEELFPRYTSLRPGPPLNPPDFQGLRDALDSEHTRRKHCERHIQSLQTRVLELQQQLAVAVAADRKKDTMIEQLDKTLARVVEGWNRHEAERTEVLRGLQEEHQAAELTRSKQQETVTRLEQSLSEAMEALNREQESARLQQRERETLEEERQALTLRLEAEQQRCCVLQEERDAARAGQLSEHRELETLRAALEEERQTWAQQEHQLKEHYQALQEESQAQLEREKEKSQREAQAAWETQHQLALVQSEVRRLEGELDTARRERDALQLEMSLVQARYESQRIQLESELAVQLEQRVTERLAQAQESSLRQAASLREHHRKQLQDLSGQHQQELASQLAQFKVEMAEREERQQQVAEDYELRLAREQARVCELQSGNQQLEEQRVELVERLQAMLQAHWDEANQLLSTTLPPPNPPAPPAGPSSPGPQEPEKEERRVWTMPPMAVALKPVLQQSREARDELPGAPPVLCSSSSDLSLLLGPSFQSQHSFQPLEPKPDLTSSTAGAFSALGAFHPDHRAERPFPEEDPGPDGEGLLKQGLPPAQLEGLKNFLHQLLETVPQNNENPSVDLLPPKSGPLTVPSWEEAPQVPRIPPPVHKTKVPLAMASSLFRVPEPPSSHSQGSGPSSGSPERGGDGLTFPRQLMEVSQLLRLYQARGWGALPAEDLLLYLKRLEHSGTDGRGDNVPRRNTDSRLGEIPRKEIPSQAVPRRLATAPKTEKPPARKKSGHPAPSSMRSRGGVWR

SEQ ID NO: 3 - DNA, 3784 bp, Artificial Sequence
  Chemically Synthesized
gtggtctcgcgggcgggtgacgtcatcgaggcgagagaggcggagccgtggacagtgggcgggaggctgccaacggttttgagcgtagggggaggcgtgagagggggatctcaggggaggaggtcaatcgcttgccccccactttggcaaattggggactgaggactggaagggtggagagtaggcggaaccaggtggtcgtcggggcagaggatctcgggctaggcttgagggcggcgtgcttcttagggacgacttagggcgtgactgagggttcacaaggtttcttttggggtggtcgggagggagagattctagggaacaaggaagctcgctatggctttcttgccaggaggggtcgaagggaaagtacaagggagctgaccctgggtagaacgggtgaagggatgggggagcgtgaggttccgccctctcttgagactggaaccaattgagggactagtagggcagggggacagaaattgggctcctagtggatttgggtccgtttccgttgggacgttttgggtgtgagaacttaagagctcagttgaccggggatagcctgtgccggagttgatctgcagcttccagcactcgtagtcgggaagaggagcttcagcagcgctgttgtcccacagtaggtcttctgtccgcacccgctctgcgctgcaccctcttaacgctgttcccaggagctggggaaagggatgcttttgcccactcccatggcccctggaactggtggaaacctttcctctaaccagaaagcctcgatatccttaattcaccaaggatccttggcgtggagtcttcctcccttctcccaagtctttctccgtgaacttttcctcctggactttgctaaagcagaacctcccagctctttgctgtctccggttgtctcttccctgtattcatggcaacatcagctgacagccccagttcacccctcggggcggaggatctcctgagtgattcatcagaaccccctgggctcaaccaagtgtcgtctgaagtgacctcccagctctatgcttctttgcgcctcagccggcaggcggaggccacggcccgagcccagctgtatttaccctccacctccccgcctcatgaagggttagacggcttcgcccaagaattgagtcgaagcttgtcagtcggattggaaaagaacttgaagaaaaaggatggttctaagcatatctttgagatggaaagtgttcggggtcagctccagaccatgctccaaacctcacgtgatacagcctatcgggatcctctcattcctggcgctggctcagagagacgggaagaggactcctttgacagtgatagcacagccaccttgctcaacacccggcccctgcaagacttgtctccatctagctcagcccaagccctggaggagctgtttccccgctacaccagccttcggccagggcctccactcaatcccccagattttcaggggctgagagatgcattggattcagagcatacccgccgcaagcattgtgagcgccatattcagagcctgcagacccgagtgttagagctacagcaacaattagccgtggctgtggctgccgaccgcaagaaagataccatgattgaacaactggacaagaccctggcccgtgtggtggagggctggaaccggcatgaggctgagcggacagaggttctcaggggacttcaagaggaacaccaggcagcagagctcaccagaagcaagcagcaggagacagtaacccgcctggaacaaagcctttctgaggccatggaggccctgaatcgtgagcaggaaagtgccagactgcagcaacgggaaagagagacactggaggaggaaaggcaagctctgactctgaggttggaggcagaacagcagcggtgctgtgtcctgcaggaagagcgggatgcagctcgggctgggcaactgagtgagcatcgagagttggagactcttcgggctgccctagaagaagaacggcagacctgggcccagcaagagcaccagcttaaggaacactaccaggcgctgcaggaggagagccaggctcagctggaaagggagaaggagaagagccagagggaagcccaggccgcctgggagacccagcaccagttggcattggtgcagtctgaggtgcggcggctggaaggagagctggatacagctcggagagagagagatgccctgcagctggaaatgagcttggtgcaggcccggtatgaaagccagcggatccagctggagtcggagctggctgtgcagctggagcagcgggtgacagagcggctggcgcaggctcaggagagcagcctacggcaagcagcctccctcagggaacatcacaggaagcagctgcaggacctgagtggacagcaccagcaggagctggccagtcagctagctcagttcaaggtggaaatggcagaacgagaggaacggcaacagcaggtggctgaggactacgagctcagactggcccgggagcaagcgcgagtgtgcgaactgcagagtgggaaccagcagctggaggagcagcgggtggagctggtggaaagactgcaggccatgctgcaggcccactgggatgaggccaaccagctgctcagcaccactctcccgccgcccaaccctccagctcctcctgctggaccctccagccccgggcctcaggagcccgagaaggaggagaggagggtctggactatgcctcccatggccgtggccctgaagcctgtattgcagcagagccgggaagcaagggacgagctacctggagcgcctcctgttctttgcagttcctcctcagatcttagcctcctgttgggcccctcttttcagagccagcattctttccagcccctggagcccaaaccagacctcacttcatccacagctggggccttctctgcacttggggccttccatcccgatcatagggcagaaaggccattccctgaggaagatcctggacctgacggggagggcctcctaaagcaagggctgccgcctgctcagctggagggcctcaagaattttttgcaccagttgctggagacagtgccccagaacaatgagaacccttctgtcgacctgttgccccctaagtctggtcctctgactgtcccatcttgggaggaagcccctcaagtgccacgtattccaccgcctgtccacaaaaccaaagttcccttagccatggcatccagtcttttccgggtccctgagcctccctcctcccattcacaaggcagtggtcccagcagtggttccccagagagaggtggagatgggcttacattcccaaggcagctgatggaggtgtctcaactgttgcgactctaccaggctcggggctggggggctctgcctgctgaggatctcctgctctacctgaagaggctggaacacagcgggtacaagcctgggaggaaggaggaaggattctccgggtggaagctggattatggggagtggagtgggactgatggccgaggggataatgtccccagaaggaacacagactcccgcttgggtgagatcccccggaaagagattccctcccaggctgtccctcgccgccttgctacagcccccaagactgaaaaacctcccgcacggaagaaaagtgggcaccctgccccgagtagcatgaggagccgggggggagtctggagatgagcccccctaccctctctcctctttgttctctcattgttgttattttaataaatgctcattagtctgcaaaaaaaaaaaaaaaaaaaa

SEQ ID NO: 4 - protein, 925 aa, Artificial Sequence
  Chemically Synthesized
MATSADSPSSPLGAEDLLSDSSEPPGLNQVSSEVTSQLYASLRLSRQAEATARAQLYLPSTSPPHEGLDGFAQELSRSLSVGLEKNLKKKDGSKHIFEMESVRGQLQTMLQTSRDTAYRDPLIPGAGSERREEDSFDSDSTATLLNTRPLQDLSPSSSAQALEELFPRYTSLRPGPPLNPPDFQGLRDALDSEHTRRKHCERHIQSLQTRVLELQQQLAVAVAADRKKDTMIEQLDKTLARVVEGWNRHEAERTEVLRGLQEEHQAAELTRSKQQETVTRLEQSLSEAMEALNREQESARLQQRERETLEEERQALTLRLEAEQQRCCVLQEERDAARAGQLSEHRELETLRAALEEERQTWAQQEHQLKEHYQALQEESQAQLEREKEKSQREAQAAWETQHQLALVQSEVRRLEGELDTARRERDALQLEMSLVQARYESQRIQLESELAVQLEQRVTERLAQAQESSLRQAASLREHHRKQLQDLSGQHQQELASQLAQFKVEMAEREERQQQVAEDYELRLAREQARVCELQSGNQQLEEQRVELVERLQAMLQAHWDEANQLLSTTLPPPNPPAPPAGPSSPGPQEPEKEERRVWTMPPMAVALKPVLQQSREARDELPGAPPVLCSSSSDLSLLLGPSFQSQHSFQPLEPKPDLTSSTAGAFSALGAFHPDHRAERPFPEEDPGPDGEGLLKQGLPPAQLEGLKNFLHQLLETVPQNNENPSVDLLPPKSGPLTVPSWEEAPQVPRIPPPVHKTKVPLAMASSLFRVPEPPSSHSQGSGPSSGSPERGGDGLTFPRQLMEVSQLLRLYQARGWGALPAEDLLLYLKRLEHSGYKPGRKEEGFSGWKLDYGEWSGTDGRGDNVPRRNTDSRLGEIPRKEIPSQAVPRRLATAPKTEKPPARKKSGHPAPSSMRSRGGVWR

SEQ ID NO: 5 - DNA, 21 bp, Artificial Sequence
  Chemically Synthesized
agugccagacugcagcaactt

SEQ ID NO: 6 - protein, 21 aa, Artificial Sequence
  Chemically Synthesized
YKPGRKEEGFSGWKLDYGEWS

SEQ ID NO: 7 - DNA, 21 bp, Artificial Sequence
  Chemically Synthesized
caacuggacaagacccuggtt

SEQ ID NO: 8 - DNA, 21 bp, Artificial Sequence
  Chemically Synthesized
ggctacgtccaggagcgcacc

SEQ ID NO: 9 - DNA, 19 bp, Artificial Sequence
  Chemically Synthesized
cagtcgcgtttgcgactgg